(19) United States
(12) Patent Application Publication
(10) Pub. No.: US 2022/0177921 A1
(43) Pub. Date: Jun. 9, 2022

(54) GENE THERAPY USING GENETICALLY MODIFIED VIRAL VECTORS

(71) Applicant: BioViva USA, Inc., Bainbridge Island, WA (US)

(72) Inventors: Elizabeth Louise Parrish, Bainbridge Island, WA (US); Anca Selariu, (US)

(21) Appl. No.: 17/680,708

(22) Filed: Feb. 25, 2022

Related U.S. Application Data
(63) Continuation-in-part of application No. 16/272,956, filed on Feb. 11, 2019, now abandoned.
Application No. 16/272,956 is a continuation-in-part of application No. 17/592,803, filed on Feb. 4, 2022.
(60) Provisional application No. 62/723,469, filed on Aug. 27, 2018.
(60) Provisional application No. 63/146,538, filed on Feb. 5, 2021.
(60) Provisional application No. 63/188,652, filed on May 14, 2021.
(60) Provisional application No. 63/305,836, filed on Feb. 2, 2022.

(51) Int. Cl. C12N 15/86; A61K 48/00

(52) U.S. Cl. CPC C12N 15/86; A61K 48/0075; C12N 2710/16143; C12N 2710/16151; C12N 2710/16171; A61K 48/0091

(57) ABSTRACT

Disclosed are methods for gene therapy by administration of genetically modified viral vectors. Gene therapy vectors can include a cytomegalovirus vector encoding one or more therapeutic donor genes such as human telomerase reverse transcriptase (hTERT). These vectors can be used in exemplary gene therapy methods for maintaining or improving one or more aspects of a recipient's physiological wellness and/or longevity. The recombinant viral vector can be administered or received intranasally or as an injectable therapeutic

### CROSS-REFERENCE TO RELATED APPLICATIONS

This application is a continuation-in-part of U.S. application Ser. No. 16/272,956 titled “NOVEL METHOD FOR GENE THERAPY USING INTRANASAL ADMINISTRATION OF GENETICALLY MODIFIED VIRAL VECTORS” and filed on Feb. 11, 2019, which claims the benefit of and priority to U.S. Provisional Patent App. No. 62/723,469, titled “NOVEL METHOD FOR GENE THERAPY USING INTRANASAL TRANSFECTION OF HCMV” and filed on Aug. 27, 2018.

This application is also a continuation-in-part of U.S. application Ser. No. 17/592,803 titled “SYSTEMS AND METHODS FOR GENE THERAPY VIA ADMINISTRATION OF GENETICALLY MODIFIED VIRAL VECTORS” and filed Feb. 4, 2022, which claims the benefit and priority to: U.S. Provisional Patent App. No. 63/146,538 filed Feb. 5, 2021; U.S. Provisional Application No. 63/188,652 filed May 14, 2021; and U.S. Provisional Application No. 63/305,836 filed Feb. 2, 2022.

Each of the foregoing applications is incorporated herein by reference in its entirety.

### BACKGROUND

### Technical Field

The disclosure relates to the field of gene therapy, specifically to the field of low-cost designable gene therapy treatments.

### Related Technology

Progress in the study of genetics and cellular biology over the past three decades has greatly enhanced our ability to describe the molecular basis of many human diseases. Molecular genetic techniques have been particularly effective. These techniques have allowed for the isolation of genes associated with common inherited diseases that result from a lesion in a single gene such as ornithine transcarbamylase (OTC) deficiency, cystic fibrosis, hemophilias, immunodeficiency syndromes, and others—as well as those that contribute to more complex diseases such as cancer. Therefore, gene therapy, defined as the introduction of genetic material into a cell in order to either change its phenotype or genotype, has been intensely investigated over the last few decades.

It is currently the case that gene therapy for patients is exceedingly expensive and limited in implementation, allowing only a small number of genes or only a single gene to be inserted into a patient through a single delivery method. It is further the case that there is no commercially viable method to extend the telomeres at the ends of chromosomes in human cells, in an effort to extend lifespans through the use of telomerase. If a patient requires multiple genes or pieces of genetic code inserted for medicinal purposes, for example to extend telomeres at the end of a cell and to also protect against certain genetic disorders (or even remove them entirely, should such genetic structuring become available in the future), they may have to have gene therapies administered multiple times or in multiple different ways. Not only does this raise the already lofty cost of gene therapies, many gene therapy techniques may not quickly or effectively achieve full saturation in a patient's body. This added time is, yet another, cost a patient must incur to obtain gene therapy.

Further, so-called monogenetic traits are rare in most complex organisms. Generally, genetic traits and issues are caused by numerous genetic factors in conjunction with external factors. And, while external factors may be accounted for and dealt with adequately in many cases by modern medical practice, dealing with genetic factors, much less numerous genetic factors such as multiple gene mutations working to cause numerous issues in a patient, still pose problems in both efficacy and cost to patients. For example, the lifespan of an individual could theoretically reach as much as 120 years with appropriate, effective, and affordable gene therapy techniques, as well as countering many genetic disorders and diseases or even curing some incurable disorders or diseases in the human species.

Prior efforts toward achieving a better gene therapy system have included the use of a multiple adenoviral vector systems to transiently transduce cells to produce retroviral progeny. An adenoviral vector encoding a retroviral backbone (the long terminal repeats (LTRs), packaging sequence, and a reporter gene) and another adenoviral vector encoding all of the transacting retroviral functions (the CMV promoter regulating gag, pol, and env) has been shown to accomplish in vivo gene transfer to target parenchymal cells at high efficiency, rendering them transient retroviral producer cells. Athymic mice xenografted orthotopically with the human ovary carcinoma cell line SKOV3, and then challenged intraperitoneally with the two adenoviral vector systems, demonstrated the concept that adenoviral transduction had occurred with the in situ generation of retroviral particles that stably transduced neighboring cells in the target parenchyma. While these experimental systems have established the foundation that adenoviral vectors may be utilized to render target cells transient retroviral vector producer cells, they are unlikely to be easily amenable to clinical applications that demand reproducible, certified vector preparation because of the stochastic nature for multiple vector transduction of single cells in vivo.

Adenovirus-associated viruses are simple DNA containing viruses often requiring the function of other viruses (e.g. adenoviruses or herpes viruses) for complete replication efficiency. The virion is composed of a replication (rep) and capsid (cap) gene flanked by two inverted terminal repeats (ITRs). These vectors have the ability to integrate into a cellular genome for stable gene transfer. However, a major hinderance to further use of these vectors has been the ability to produce them in large-scale in vitro. The major obstacles to this endeavor are the toxic cellular effects of the rep and needed helper-virus genes. Examples of production methods for AAV vectors include co-transfection of plasmids delivering the ITR flanked gene of interest with a rep-cap expression cassette and the helper-virus genes (ref) and co-delivery of the ITR-flanked gene of interest along with helper-virus genes to cells stably expressing rep-cap, delivery of a chimeric virus vector, such as a herpes virus vector, with all the necessary components. Another efficient method is to deliver all the required elements in a single plasmid vector.

Deficiencies in the art regarding methods of utilizing adenoviral, retroviral, and adeno-associated elements for stable delivery of a therapeutic gene include lack of a single vector. The requirement for multiple vectors dictates that more antibiotics are used, which is more costly and furthermore undesirable, given the increasing number of strains which are becoming resistant to commonly used antibiotics. In addition, the use of multiple vectors gives reduced efficiency, since more than one transduction event into an individual cell is required, which statistically occurs at a reduced amount compared to the requirement for one transduction event. Other notable deficiencies in the art include rapid clearance and host immunity to the viral vectors, limiting their prolonged use, possible oncogenic effects from viral integration, limited cellular tropisms, and the limited size and/or number of genes that can be delivered in a single vector.

Accordingly, there are a number of disadvantages with gene therapy approaches that can be addressed to solve a long-felt and unmet need in the art.

### SUMMARY

A method that allows for multi-gene cassettes to be inserted easily into a patient is disclosed, which may allow for multi-genetic treatments, important for many health issue and potential life-changing effects. A method to effectively, long-lastingly, and affordably insert one or more donor sequences into a host body for therapeutic purposes would achieve this. A human cytomegalovirus (“HCMV”) or varicella zoster virus (“VZV”) would achieve the goals of viable single or multi-gene insertion, and be reactivatable in a host body. Beneficially, the virus (HCMV or VZV) itself is asymptomatic in non-immunocompromised individuals, making it an optimal delivery method for affordable, effective gene therapy.

Accordingly, in a preferred embodiment, a novel method for gene therapy comprises administration of genetically modified viral vectors such as HCMV or VZV. The following non-limiting summary of the invention is provided for clarity, and should be construed consistently with embodiments described in the detailed description below.

To solve the problem of a lack of a cheap and effective system for single or multi-gene insertion into target host cells for gene therapy, a novel method for gene therapy using administration (e.g., injection or intranasal delivery) of genetically modified viral vectors has been devised, utilizing techniques for viral transfection (also known as viral transduction) of cells, polymerase chain reactions (PCR) to grow desired genetic components, utilizing a virus (e.g., HCMV and/or VZV) for transcription of one or more donor sequences into a host body. In some embodiments, target genes may be selected for a given patient's therapy, prepared through bacterial artificial chromosome (BAC) growth and PCR techniques and viral transduction. A solution or other composition may be prepared containing viruses and/or viral vectors with the desired genes for administration to a patient via an appropriate route such as intranasally or by injection.

Disclosed is a novel method for gene therapy through administration of genetically modified viral vectors, including: selecting target genes for a patient's gene therapy; using polymerase chain reactions to create at least one desired gene, where the desired gene contains at least one recombination site(s); wherein a desired gene may be extracted from a bacterial, animal, or plant cell, or virus; creating an empty coding sequence consisting of identical recombination sites to those of the desired genes; wherein an empty coding sequence also contains a leading and trailing untranslated region; recombining a desired target gene into an empty coding sequence, thereby creating a full genetic coding sequence capable of creating proteins in a cell; wherein recombination of a desired target gene takes place in a bacterial cell; sequencing bacterial DNA to confirm that the desired gene is present in the bacterial DNA; performing viral transduction to infect a desired virus with the sequenced target gene; producing a solution or composition containing a sufficient amount of viral agents to introduce to a patient effectively; and administering a solution containing viruses with the desired genetic traits for infection of a human host.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 is a method diagram of high-level steps needed for intranasal multi-gene HCMV or VZV insertion into a patient's body, according to a preferred embodiment.

FIG. 2 is a method diagram of high level steps taken for generation and experimental verification of desired genes in an HCMV or VZV virus, according to a preferred embodiment.

FIG. 3 is a diagram of an HCMV cell and important components of such.

FIG. 4 is a diagram of the lifecycle of a patient's cells once infected with HCMV or VZV.

FIG. 5 is a diagram of the construction of a gene expression cassette for implementation in a HCMV or VZV for delivery into a patient.

FIG. 6 is a diagram of a partial process of double homologous recombination of genes, according to a preferred aspect.

FIG. 7 is a diagram of a partial process of double homologous recombination of genes, according to a preferred aspect.

FIG. 8 is a method diagram of gene recombination and construction of desired genes for insertion into a HCMV or VZV.

FIG. 9 is a diagram of the construction of a gene expression with a plurality of genes inserted into a single cassette, for application in a HCMV or VZV, according to a preferred aspect.

FIG. 10 is a method diagram of HCMV or VZV delivery intranasally and the process of being metabolized into the host body through the liver, according to a preferred embodiment.

FIG. 11 is a diagram of key steps in a method for bacterial gene recombination to acquire desired genes for later viral transfection.

FIG. 12 is a diagram of key steps in a method for bacterial gene recombination to acquire desired genes for later viral transfection.

FIG. 13 is a method diagram showing steps in the creation of mouse cytomegalovirus (MCMV)-Luc SW102 electro competent cells, as an alternative method of introducing desired genes into a cytomegalovirus for administration, according to a preferred aspect.

FIG. 14 is a diagram showing the insertion of an m-tert gene at a specific locus in a bacterial artificial chromosome in a mouse cytomegalovirus (MCMV).

FIGS. 15A-15E illustrate example bacterial artificial chromosome (BAC) constructs that may be utilized to generate the CMV vectors disclosed herein, the constructs including one or more therapeutic genes suitable for administration to human subjects. FIG. 15A shows an example construct that includes human telomerase reverse transcriptase (hTERT) as an exogenous insert. FIG. 15B shows an example construct that includes human follistatin-344 (hFST) as an exogenous insert. FIG. 15C shows an example construct that includes klotho (KL or KLOTHO) as an exogenous insert. FIG. 15D shows an example construct that includes Dsup as an exogenous insert, and FIG. 15E shows an example construct that includes PGC1α as an exogenous insert.

FIGS. 16A-16F illustrate example BAC constructs that may be utilized to generate the CMV vectors disclosed herein, each construct including multiple therapeutic genes suitable for administration to human subjects. FIG. 16A shows an example construct that includes both hTERT and hFST as exogenous inserts. FIG. 16B shows an example construct that includes both hFST and KLOTHO as exogenous inserts. FIG. 16C shows an example construct that includes both hTERT and KLOTHO as exogenous inserts. FIG. 16D shows an example construct that includes hTERT, hFST, and KLOTHO as exogenous inserts. FIG. 16E shows an example construct that includes hTERT, hFST, and Dsup as exogenous inserts, and FIG. 16F shows an example construct that includes hTERT, hFST, and PGC1α as exogenous inserts.

FIG. 17 illustrates a method for manufacturing a CMV-hFST recombinant BAC via homologous recombination.

FIG. 18 illustrates a method for manufacturing a CMV-hTERT recombinant BAC via homologous recombination.

FIG. 19 illustrates a method for manufacturing a CMV-hFST+hTERT recombinant BAC via homologous recombination.

FIG. 20 illustrates a method for manufacturing a CMV-hFST+hTERT+KL recombinant BAC via homologous recombination.

FIGS. 21A-21D illustrate the construction and characterization of recombinant MCMV_{TERT} and MCMV_{FS344} (also referred to herein as simply MCMV_{FST}) expressing mTERT and mFS344 (also referred to herein as simply mFST). FIG. 21A is a graphical representation of the location of mTERT and mFS344 genes insertion in the MCMV genome. FIG. 21B illustrates a Western blot analysis of mTERT and mFS344 in MCMV_{TERT} and MCMV_{FS344} infected cells as compared to cells infected with respective non-recombinant MCMV (WT). FIG. 21C illustrates a growth curve of MCMV_{TERT} and MCMV_{FS344} viruses in cell culture as compared to wild-type, non-recombinant MCMV (WT). FIG. 21D illustrates images obtained using IVIS during an in vivo luciferase assay of 20 months old C57BL/6J mice that were uninfected, infected with wild-type MCMV virus, or infected with MCMV_{TERT} or MCMV_{FS344} recombinant virus.

FIGS. 22A-22D are various graphs of data illustrating improved health of mice infected with recombinant MCMV_{TERT} or MCMV_{FS344} virus. In particular, FIG. 22A illustrates the results of a glucose tolerance test (GTT) in mice treated intraperitoneally (IP) or intranasally (IN) with WT-MCMV, with recombinant MCMV_{TERT} virus, or with recombinant MCMV_{FS344} virus, as compared to untreated (UN) controls. FIG. 22B is a bar graph illustrating the calculated area under the curve (AUC) in the GTT assay of FIG. 22A. FIG. 22C is a bar graph illustrating the results of a 3-minute beaker test in the respective groups, and FIG. 22D is a bar graph illustrating the results of a beam test for mice tested in each infected and uninfected group.

FIG. 23A is a graph illustrating mTERT mRNA levels within different organ tissues of mice 5 days post infection with MCMV_{Luc-TERT}.

FIG. 23B is a graph comparing TERT and FST mRNA levels in different organs of WT, MCMV_{TERT}, and MCMV_{FST} treated mice by qPCR, with fold increase in MCMV_{TERT}, and MCMV_{FST} treated mice over WT treated mice indicated numerically on the top of each bar.

FIG. 23C illustrates the determination of relative telomere length in different organs of treated and untreated groups at 24-months-old mice. An 8-months-old mouse was also measured. The qPCR was performed on the genomic DNA using specific telomeric primers. A pair of 36B4 gene primers was used as a single copy gene for normalization.

FIGS. 24A-24C illustrates detection of mTERT and mFST recombinant proteins by ELISA in the sera of WT, MCMV_{TERT}, and MCMV_{FST} treated (IP or IN) 24-month-old mice. FIGS. 24A and 24B illustrate detected levels over four days. FIG. 24C illustrates detection of recombinant mTERT by ELISA over a one-month period of time, as detected in the serum of 8-month-old mice infected or uninfected with recombinant MCMV_{TERT} virus. In each of FIGS. 24A-24C, mock treated mice were used as negative control. Each data point represents the average value of three mice. Error bars represent standard deviations.

FIGS. 25A-25D provide evidence that treatment with recombinant MCMV_{TERT} and MCMV_{FS344} virus improves quality of life in treated mice. FIGS. 25A and 25B are exemplary images of 26-month-old C57BL/6J mice exhibiting hair loss as an uninfected control (FIG. 25A) and following infection by wild-type MCMV virus (FIG. 25B). FIGS. 25C and 25D are exemplary images of 26-month-old C57BL/6J mice exhibiting no loss of hair (or hair retention) following infection with recombinant MCMV_{TERT} (FIG. 25C) and MCMV_{FS344} (FIG. 25D) virus.

FIG. 26 illustrates a graph of the average body weight of mice in the untreated and treated cohorts; each cohort being tested within two different treatment sets.

FIGS. 27A and 27B illustrates survivorship for mice in the untreated and treated cohorts; each cohort being tested within two different treatment sets.

FIGS. 28A-28C illustrate an analysis of mouse heart muscle by electron microscopy. FIG. 28A illustrates electron micrographs of heart muscle taken from 6-month old mice (young) and from aging mice within the uninfected control (UN), wild-type-MCMV-infected control (MCVM), MCMV_{TERT}-infected cohort, and MCMV_{FS344}-infected cohort (scale bar=500 nm). From these micrographs (N=20 in each group), the number of mitochondria within connected cristae and the density of mitochondria within mouse heart muscle is calculated (FIGS. 28B and 28C, respectively). Values are mean±S.E., p<0.05 using the unpaired Student's t-test.

FIGS. 29A-29C illustrate an analysis of mouse skeletal muscle by electron microscopy. FIG. 29A illustrates electron micrographs of skeletal muscle taken from 6-month old mice (young) and from aging mice within the uninfected control (UN), wild-type-MCMV-infected control (MCVM), MCMV_{TERT}-infected cohort, and MCMV_{FS344}-infected cohort (scale bar=500 nm). From these micrographs (N=20 in each group), the number of mitochondria within connected cristae and the density of mitochondria within mouse skeletal muscle is calculated (FIGS. 29B and 29C, respectively). Values are mean±S.E., p<0.05 using the unpaired Student's t-test.

FIG. 30 illustrates the results of mouse skeletal muscle tissue homogenates obtained from uninfected mice and mice infected with recombinant MCMV expressing mouse TERT and mouse FS344 genes and probed by Western blot for various mitochondrial and autophagy markers. PGC1α and TFAM represent transcriptional factors for mitochondrial biogenesis; Complex I, Complex II, Complex III, and Complex V represent markers for mitochondria function; and LCI, LC3II, and p62 represent autophagy markers. GAPDH was used as a loading control.

FIG. 31 illustrates the results of mouse heart muscle tissue homogenates obtained from uninfected mice and mice infected with recombinant MCMV expressing mouse TERT and mouse FS344 genes and probed by Western blot for various mitochondrial and autophagy markers. PGC1α represents transcriptional factors for mitochondrial biogenesis; Complex I, Complex II, Complex III, and Complex V represent markers for mitochondria function; and LC3I, LC3II, and p62 represent autophagy markers. GAPDH was used as a loading control.

### DETAILED DESCRIPTION

Disclosed are methods that allow for single or multi-gene cassettes (used synonymously herein with “payloads,” “inserts,” and the like) to be inserted easily into a patient. For example, a human cytomegalovirus (“HCMV”) or varicella zoster virus (“VZV”) could be used to deliver the multi-gene cassette that may be reactivatable in a host body. In other words, the gene cassette may be delivered, read and transcribed in the host body. Beneficially, the HCMV or VZV themselves are asymptomatic in non-immunocompromised individuals, making them optimal delivery methods for affordable, effective gene therapy.

Surprisingly, treatment methods disclosed herein that include administration of recombinant viral vectors demonstrated several beneficial effects. For example, MCMV_{TERT} virus and recombinant MCMV_{FS344} (also referred to herein as MCMV_{FST}) virus demonstrated increased serum expression of TERT and FS344 gene products, respectively, and also increased healthy life span of the subject by up to 40%. Both viral vectors demonstrated increased autophagy and increased number and concentration of mitochondria in skeletal and heart muscle. The disclosed treatments additionally increased physical coordination, increased glucose tolerance, and prevented age-related hair loss—none of which were expected benefits of the disclosed gene therapies. Embodiments of the present disclosure can have multiple applications: preserving the health and muscle of astronauts during low orbit and deep space missions; cost reduction in insurance because of delayed need for care for chronic conditions; possible improvement in type-2 diabetes; and possible protective effects in chronic inflammatory conditions. Many of these are significant risk factors for exaggerated immune reactions during infections with common pathogens. In addition to human health, such therapies could have application in animal health, increasing the lifespan of pets and/or the health of livestock animals, for example. It is also envisioned that embodiments disclosed herein will be utilized in research applications in which the “subject” is a laboratory animal such as a mouse, non-human primate (e.g., rhesus macaque), or other laboratory mammal.

### Viral Vectors

As provided above, there are a number of disadvantages with current gene therapy approaches that can be addressed to solve a long felt and unmet need in the art. In particular, none of the traditional retroviral, adenoviral, or adeno-associated gene therapy systems are capable of long-term, full-body expression of their payload in a manner that can promote the maintenance or improvement of aspect(s) of the recipient's physiological wellness and/or longevity. Instead, most, if not all, of the aforementioned gene therapy systems are directed to permanent correction of physiological or genetic defects within the targeted cells (e.g., via integration of the genetic payload at the target site) or are otherwise engineered to target tumors or provide cancer therapies. There is an apparent lack of solutions that address the many symptoms, effects, and/or disorders associated with aging.

Embodiments of the present disclosure solve one or more of the noted problems in the art. For example, a recombinant CMV vector is disclosed that encodes the TERT gene, the Follistatin (FS344) gene (also referred to herein as FST), and/or any of the other genes disclosed herein, and these therapeutic tools can be used to promote the maintenance or improvement of a recipient's physiological wellness and/or longevity. In all embodiments disclosed herein, it will be understood that a recombinant varicella zoster virus (VZV) may be used as an alternative to the recombinant CMV, or vice versa.

The disclosed recombinant vectors can be safely administered to a patient (e.g., intranasally and/or as an injectable preparation) and thereby deliver gene therapy to multiple organs with long-lasting benefits and no carcinogenicity or any other observable detrimental side effects. As a result, the disclosed vectors and associated treatment methods can increase longevity of patients by at least 10%, preferably at least 20%, and in some embodiments, by at least 30%. Additionally, the disclosed treatment methods can surprisingly and unexpectedly increase blood glucose tolerance, retain, or increase muscle mass, increase physical coordination, increase mitochondrial biogenesis in heart and skeletal muscle, increase autophagy, promote hair retention (or prevent age-related hair loss), or combinations thereof.

CMV and/or VZV are good candidate viruses for recombinant viral vectors because they have a large genome with a unique ability to incorporate multiple additional and/or different genes. Further, CMV and/or VZV viral vectors are capable of delivering the multiple additional and/or different genes to a host body without infecting the host genome with viral genetic material (i.e., the vial genome of CMV and/or VZV is not passed to or shared with the host). Further, these viruses generally do not illicit an immune response from the host body. Human CMV (hCMV) and mouse CMV (mCMV) have both seen success in human clinical trials as safe and efficient viral vectors.

For example, the disclosed recombinant vectors can be safely administered to a patient (e.g., intranasally and/or as an injectable preparation) and thereby deliver gene therapy to multiple organs with long-lasting benefits and no carcinogenicity or any other observable unwanted side effects. As a result, the disclosed vectors and associated treatment methods can increase longevity of patients by at least 10%, preferably at least 15%, preferably at least 20%, 25%, 27%, 29% and in some embodiments, by at least 30%. Additionally, the disclosed treatment methods can, surprisingly and unexpectedly, increase blood glucose tolerance, retain or increase muscle mass, increase physical and neuromuscular coordination, increase mitochondrial biogenesis in heart and skeletal muscle, increase autophagy, promote hair retention (or prevent age-related hair loss), or combinations thereof.

These salubrious effects are unexpected. For example, with respect to vector selection, CMV is a ubiquitous virus that is present in over 60% of the population. Following primary infection, CMV persists for the life span of the host, and although CMV is generally benign in healthy individuals, the virus can cause devastating disease in immunocompromised populations resulting in high morbidity and mortality. CMV is also one of the most immunogenic viruses known. High antibody titers are directed against numerous viral proteins during primary infection of healthy individuals. In addition, a large proportion of the host T-cell repertoire is also directed against CMV antigens, with 5-10-fold higher median CD4+ T-cell response frequencies to CMV than to acute viruses (e.g., measles, mumps, influenza, adenovirus) or even other persistent viruses such as herpes simplex and varicella-zoster viruses. A high frequency of CD8+ T-cell responses to defined CMV epitopes or proteins are also commonly observed. In a large-scale human study quantifying CD4+ and CD8+ T-cell responses to the entire CMV genome, the mean frequencies of CMV-specific CD4+ and CD8+ T-cells exceeded 10% of the memory population for both subsets, and in some individuals, CMV-specific T-cells account for >25% of the memory T-cell repertoire.

Paradoxically, the robust immune response to CMV is unable to either eradicate the virus from healthy infected individuals or confer protection against re-infection. This ability of CMV to escape eradication by the immune system and to re-infect the seropositive host has long been believed to be linked to the multiple viral immunomodulators encoded by the virus. As a result, CMV-based vectors expressing heterologous antigens do not induce cytotoxic T-cells directed against immunodominant epitopes of those heterologous antigens. This is particularly disadvantageous if the CMV-based vector is engineered as a vaccine or tumor-specific therapy as it limits the efficacy of the T-cells raised by such a CMV-based vaccine to protect against infection by a pathogen or to otherwise mount a cellular immune response against a tumor. On the other hand, while CMV-based vectors are poor candidates for vaccine development, the host's continued susceptibility to infection provides a unique opportunity for the serial administration of gene therapy—insofar as the payload is not toxic and the vector is not carcinogenic.

### Vector Payloads

Telomeres are short-repeated DNA segments (for example, 5′-TTAGGG-3′ in vertebrates) at chromosome ends which are incompletely replicated during cell divisions. They protect our genetic material by acting as a chromosome cap, keeping them from binding to each other or breaking down. Once telomeres become too short, cells cease dividing and undergo apoptosis. The telomerase enzyme is responsible for adding nucleotide base pairs on chromosome ends to maintain telomere length. Telomerase is highly expressed in tissues that undergo constant and/or rapid cell division, such as cells in germline tissue, bone marrow, and linings in the gastrointestinal tract. In contrast, telomerase is usually undetectable or minimally active in mitotic or less active tissues, which results in shorter telomeres with each cell division and leads to the accumulation of senescent cells. The accumulation of senescent cells manifests as aging in an organism. A rare autosomal dominant mutation in the gene that codes for the RNA component of telomerase causes premature aging and death, most often from infections related to bone-marrow failure. Because telomerase maintains cell proliferation and division by reducing the erosion of chromosomal ends, mice deficient in TERT, like humans with defective TERT, have shorter telomeres and a shorter life span.

Telomere shortening is observed in every individual with old age. It has been found that people 60-years of age or older that have shorter telomeres have three (3) times higher risk of getting heart disease, and longer telomeres are positively correlated to a longer lifespan. Despite this, there are no data to substantiate telomere length as being related to an increased risk of heart disease or for enabling a longer lifespan.

Recent studies have shown that expressing exogenous TERT in a mammal can revert the hyper-aging process caused by a TERT deficiency, suggesting a direct role of TERT in the aging process. This also suggests that levels of TERT expressed in a mammal is a delicate balancing act over time as the mammal ages.

The degradation of telomeres in humans has been linked to shorter lifespans, cancers, and certain genetic disorders such as cri du chat, a disorder which causes numerous problems for affected children, and is caused by partial deletion of the short arm of chromosome 5. The absence of human TERT (hTERT) is heavily associated with the cri du chat disorder, and may be preventable with the disclosed techniques.

Follistatin (FS344) is a monomeric secretory protein expressed in nearly all tissues. In muscle cells, follistatin functions as a negative regulator of myostatin—a myogenesis inhibitory signal protein. Follistatin is thus known to increase skeletal muscle mass, and studies have suggested that it neutralizes the effect of various TGF-βligands, including myostatin, and activin inhibition complex through a binding to Act RIM receptor. Follistatin gene knockout mice have retarded growth, skeletal defects, reduced body mass, and die in a few hours after birth, suggesting an important role of follistatin in skeletal and muscle development. Transgenic mice expressing an enhanced level of follistatin show increase muscle mass by 194-327%, suggesting a direct role of follistatin in body mass development. Mice lacking the myostatin gene have reduced number of muscle fibers and smaller muscle fiber size. Myostatin inhibits the expression of myoD and Pax-3, the transcriptional regulators of body mass development, which results in low body mass and small-fiber size. These findings strongly suggest an important role of follistatin in the treatment of muscular dystrophy and other age-related diseases.

The klotho protein is a ubiquitous transmembrane protein that functions to enzymatically hydrolyze steroid β-glucuronides. Klotho plays a role in modulating insulin sensitivity, promoting binding of fibroblast growth factors to their corresponding receptors, regulating calcium homeostasis, minimizing oxidative stress and inflammation, preventing endothelial dysfunction, and promoting myelin integrity and concomitant cognitive function. There are three subtypes of klotho, referred to as α-klotho, β-klotho, and γ-klotho. Each of these subtypes are included in this disclosure and one or more klotho subtypes may be utilized as part of the recombinant CMV payload. Typically, the generic phrase klotho, if not specified otherwise, refers to the α-klotho subtype. Suboptimal levels of klotho protein are associated with degenerative processes such as akin atrophy, osteoporosis, and arteriosclerosis. Low levels of circulating klotho protein are known to be associated with aging. Transgenic mice lacking the α-klotho enzyme develop symptoms of premature aging, whereas transgenic mice that overexpress klotho live longer than wild-type mice.

The Dsup protein is believed to be unique to animals of the phylum Tardigrada, commonly referred to as tardigrades. Tardigrades are ubiquitously present throughout the Earth's biosphere, including in relatively inhospitable locations such as deep sea vents and the Antarctic. Tardigrades are among the most resilient lifeforms known, if not the most resilient. Tardigrades are known to be capable of surviving ionizing radiation at doses hundreds of times higher than lethal levels for humans, low temperatures approaching absolute zero, high temperatures approaching 150° C., pressures about 6 times higher than that found at the deepest levels of the oceans, and even the vacuum of outer space. Dsup functions to protect DNA against damage from ionizing radiation, even when tardigrades are in an active, non-desiccated state. Adding Dsup protein to cultured human cells was shown to reduce DNA damage from X-ray radiation by 40%. While the exact DNA protective mechanism of Dsup is unknown, it is believed to form protective molecular aggregates that associate with nucleosomes in the cell to shield DNA.

PGC-1-α regulates mitochondrial biogenesis and liver gluconeogenesis. Specifically, PGC-1-α is a transcriptional coactivator that functions as an integrator of external stimuli to promote transcription of genes involved in energy metabolism, including promoting the production of new mitochondria. Increased levels of PGC-1-α resulting from aerobic exercise have also been shown to increase autophagy in skeletal muscle.

Oct-4 (octamer-binding transcription factor 4; also known as POU5F1), Sox2 (sex determining region Y-box 2), and KLF4 (Kruppel-like factor 4) are transcription factors that function to activate or deactivate certain expressions related to stem cell differentiation. These proteins play a role in maintaining the pluripotency of certain stem cells, and lower levels promotes stem cell differentiation. These and other similar factors may also be capable of inducing pluripotency, with potential applications for regenerative medicine.

Donor genes described herein may synonymously be referred to as exogenous genes, exogenous donor genes, insert genes, and the like. Donor genes may be referred to by the name of the gene itself, or by the name of the protein for which the gene encodes. For example, the Oct-4 protein is technically encoded by the POU5f1 gene, but it will be understood that the same gene may be referred to herein as the Oct-4 gene.

### Methods of Manufacture and Use

Embodiments of the present disclosure further provides a therapeutic composition containing the recombinant CMV virus or vector (or the recombinant VZV virus or vector) and a pharmaceutically acceptable carrier or diluent. The therapeutic composition is useful in the gene therapy and immunotherapy embodiments of the present disclosure, e.g., in a method for transferring genetic information to an animal or human in need of such which may comprise administering to the host the composition; and embodiments of the present disclosure accordingly include methods for transferring genetic information.

In yet another embodiment, methods are provided for generating a recombinant viral vector (e.g., CMV) configured to express a protein, gene product, or expression product following infection or transfection of a cell in vitro or in vivo. Within an in vitro environment, embodiments of the present disclosure provide methods for cloning or replicating a heterologous DNA sequence which may comprise infecting or transfecting a cell in vitro with a recombinant CMV virus or vector disclosed herein and optionally extracting, purifying, and/or isolating the DNA from the cell or progeny virus.

Embodiments of the present disclosure provide, in another aspect, a method for preparing the recombinant CMV viruses or vectors disclosed herein, which may comprise inserting the exogenous DNA into a non-essential region of the CMV genome. The method can further include deleting a non-essential region from the CMV genome, preferably prior to inserting the exogenous DNA.

The methods provided herein can include in vivo and/or in vitro recombination. Thus, methods of the present disclosure can include transfecting a cell with CMV DNA into a cell-compatible medium in the presence of donor DNA, which may comprise exogenous DNA flanked by DNA sequences homologous with portions of the CMV genome, whereby the exogenous DNA is introduced into the CMV genome, and optionally then recovering CMV modified by the in vivo recombination.

The method can also include cleaving CMV DNA to obtain cleaved CMV DNA, ligating the exogenous DNA to the cleaved CMV DNA to obtain hybrid CMV-exogenous DNA, transfecting a cell with the hybrid CMV-exogenous DNA, and optionally then recovering CMV modified by the presence of the exogenous DNA.

Since in vivo recombination is enabled, embodiments of the present disclosure accordingly also provide a plasmid and/or bacterial artificial chromosome (BAC) system which may comprise donor DNA not naturally occurring in CMV encoding a polypeptide foreign to CMV, the donor DNA being provided within a segment of CMV DNA which would otherwise be co-linear with a non-essential region of the CMV genome such that DNA from a non-essential region of CMV is flanking the donor DNA.

The exogenous DNA can be inserted into CMV to generate the recombinant CMV in any orientation which yields stable integration of the exogenous DNA, and expression thereof, when desired.

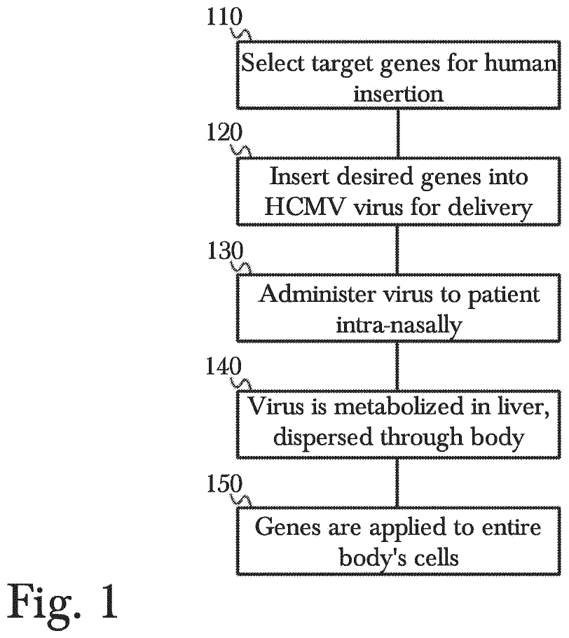

FIG. 1 is a method diagram of high-level steps needed for intranasal multi-gene HCMV or VZV viral vector administration into a patient's body, according to a preferred embodiment. First, target genes are selected for human insertion 110 according to a desired outcome or change in patient genetics, which may fluctuate and alter greatly depending on a patient's individual medical needs and their own genetic makeup. Such genes may include a TERT gene for insertion in the patient to promote telomerase reverse transcriptase enzyme production, in an effort to lengthen telomeres in a target patient. Other genes may be included as needed. The genes selected may be a plurality of genes or only one target gene, as required for a patient's therapy. The selected genes may be compiled or combined into a gene cassette and/or a multi-gene cassette.

Such genes, once selected, are inserted into the HCMV or VZV virus 120, through processes elaborated upon further in this application, including bacterial recombination and viral transfection. A composition containing the virus is inserted into one of several possible intranasal administering devices, including a dropper or sprayer, and then administered to the patient 130 intranasally. Intranasal delivery allows for fast absorption into the body, where the composition and/or viral vector may then be metabolized by the liver and dispersed through the body 140. Once this occurs, based on the lifecycle of infected cells and the HCMV or VZV virus in the host body, the majority of the body's cells are then infected with whatever gene or multi-gene cassettes may be carried by the HCMV or VZV viral vector 150, thus allowing for fast and effective gene therapy, provided an HCMV or VZV virus may be programmed with selected genes 120.

HCMV or VZV may be used for the optimal delivery of gene cassettes to the majority of the body's cells. The gene cassettes inserted into the viral genome may be present in the double-stranded form when using a HCMV or VZV delivery method. In contrast, use of AAVs require single-stranded genes to be encoded in the viral genome. Due to the difference in cell entry, replication and growth, HCMV and/or VZV may not simply be switched out in place of AAV.

Beneficially, HCMV may be delivered intranasally and thus absorbed into the blood stream through the lungs. As the modified HCMV circulates through the blood stream, it may be taken up by other cells in the host body, thereby integrating throughout the entirety of the host body. When the blood stream is filtered through the liver, HCMV is beneficially ignored by the liver cells and allowed to pass through to the other cells of the host body. The modified genome of HCMV will not be integrated into the host genome, thereby remaining benign to the host. However, the modified genome of HCMV will be replicated, transcribed, and translated to deliver the genes of interest to the cells of the host body.

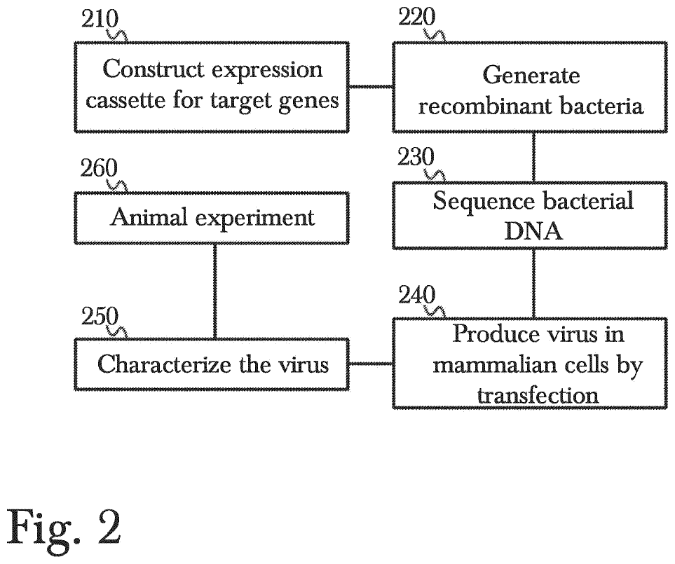

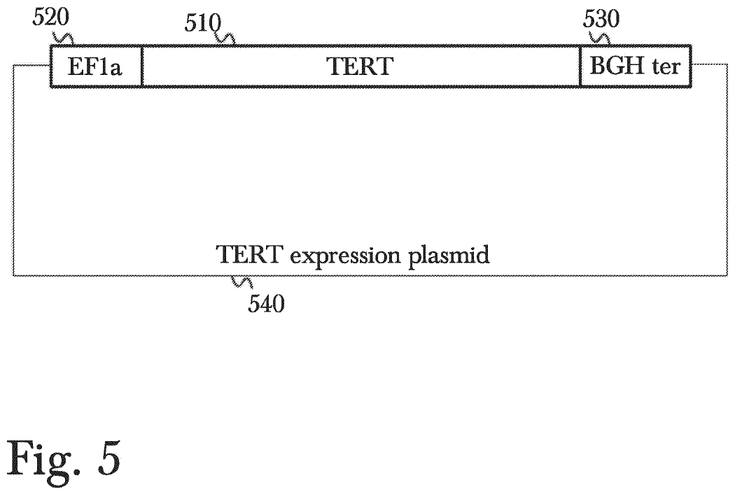

FIG. 2 is a method diagram of high-level steps taken for generation and experimental verification of desired genes in an HCMV or VZV virus, according to a preferred embodiment. First, constructing a gene or multi-gene cassette for target genes 210 must be accomplished (see FIGS. 5-8). A gene cassette may contain one or a plurality of genes, including galK gene, or hTERT or mTERT genes, and/or FST genes, which may code for multiple enzymes and proteins, depending on a patient's treatment plan.

Telomerase reverse transcriptase genes may enable the patient to produce a key enzyme in the production and maintenance of telomeres at the end of chromosomes within their cells, which may prolong lifespan and produce other benefits in a patient. Recombinant bacteria is generated 220, the purpose of which is to create specific proteins and RNA, which are important steps in the creation of gene cassettes for transfection of viral cells for later administration to a human patient. When desired bacteria are created with the desired genes, gene cassettes and gene expressions, its DNA is sequenced 230, and recombinant BACs are transfected to mammalian cells such that recombinant viruses may be produced 240. BACs are plasmids that represent constructed DNA segments which can represent specific desired genes, and can be grown and sequenced out of a bacterium, or transferred to other cells, depending on the utilization desired by the practitioner or practitioners involved. Once a virus is produced 240, the virus is characterized 250 to ensure it has the correct genetic cassettes for insertion, before it is tested on animals 260 to ensure viability and efficacy. Characterization of the virus includes isolating and sequencing the DNA in the virus to confirm the presence of the desired genes and/or multi-gene cassettes. Such a virus operates through viral transduction, a type of transfection whereby a virus may be the carrier for genetic material into host cells.

Embodiments of the present disclosure enable various treatment methods. For example, a treatment method can include a longitudinal treatment method where a plurality of therapeutically effective dosages of the composition are provided to a patient over a period of time. In some instances, the period of time is as long as 6-12 months with dosages being administered to the patient every other month, every month, every three weeks, every other week, every week, or more regularly. In an exemplary treatment method, a therapeutically effective dosage is administered to the patient every month for 8 months. In some embodiments, the patient is a middle-aged or elderly patient. For example, the patient can be a human patient that is 50 years old, 55 years old, 60 years old, 65 years old, 70 years old, or older (or can be any age falling within a range formed by the foregoing ages). The patient can also be an animal patient having an analogously middle-age or elderly age. The patient may also be younger than 50 years old, for example 40-45 years old.

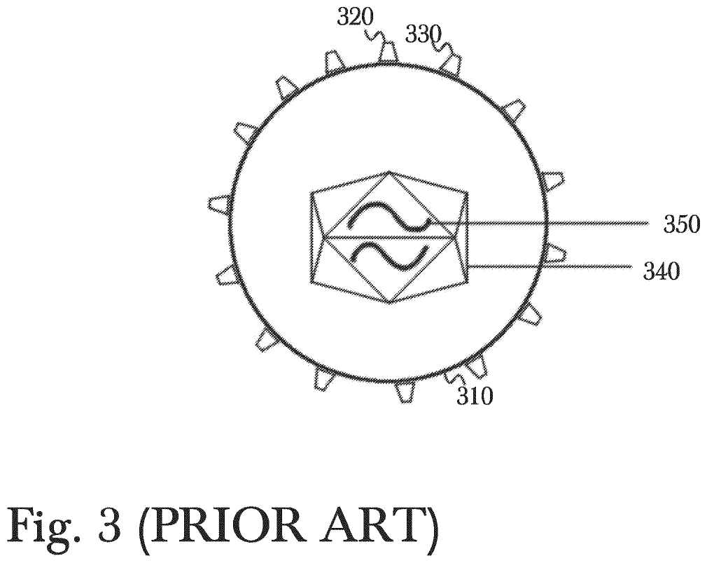

FIG. 3 is a diagram of an HCMV cell and important components of such. A viral envelope 310 exists, which may be derived partially from phospholipids and proteins common in human cells, but also contains glycoproteins 320, 330. The viral envelope 310 encapsulates a capsid 340, which is a protein shell that protects and contains the genetic material 350 of the virus. Such genetic material may include a TERT gene or other genes as desired by a healthcare practitioner for administering to a patient, for the purpose of treating disorders caused by a lack of the hTERT enzyme, rebuilding and growing the telomeres in patient cells, and more. The viral envelope 310, containing the viral capsid 340, may consist of alternating glycoproteins 320, 330 for binding to receptor sites on host cells. Inside a capsid 340 is the genome of the virus 350, containing the virus' genetic code, and also ideally containing gene cassettes built from selected genes 110 that are to be administered to a patient.

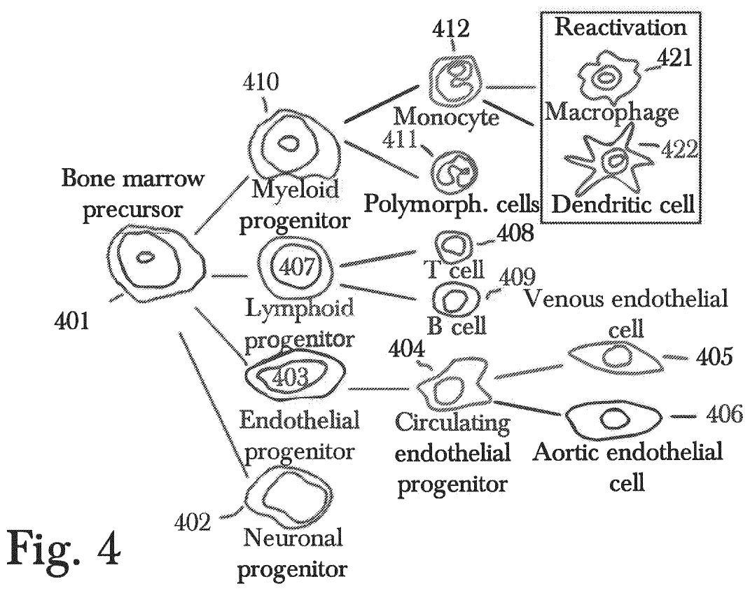

FIG. 4 is a diagram of the lifecycle of a patient's cells once infected with HCMV or VZV. A bone marrow precursor cell 401 is sometimes referred to as a type of unipotent cell which is a stem cell that has differentiated slightly and lost many of its stem cell properties. However, bone marrow precursors may still become many different types of cells in the body, and, once infected with an HCMV or VZV virus payload (containing desired genetic coding sequences), may effectively deliver these sequences to the rest of the body. A bone marrow precursor cell 401 has several lineages of transformation it may undergo, one of which being that to a neuronal progenitor 402, which has the ability to further differentiate into a finite number of neuronal and glial cell types later on.

Another lineage of transformation or differentiation that a bone marrow precursor cell 401 may undergo includes an endothelial progenitor 403, the endothelial lining being a key structural component of vasculature (i.e., blood and other vessels) inside the human body and many other animal bodies. Such a progenitor cell 403 may further differentiate into a circulating endothelial progenitor cell 404, and then possibly a venous endothelial cell 405 or an aortic endothelial cell 406. In this way, spread of the viral vector throughout the cardiovascular system is achieved. A separate lineage of differentiation for further bodily infection that is possible once bone marrow precursor cells 401 are infected is that of a lymphoid progenitor 407. The lymphoid progenitor 407 can differentiate into types of cells such as T-cells 408 and B-cells 409, key components in the body's immune system.

Another lineage of differentiation that is possible for a bone marrow precursor 401 is a myeloid progenitor 410, which may further differentiate into polymorphic cells 411 and monocytes 412, which are a form of white blood cell and capable of differentiating into a macrophage 421 or a dendritic cell 422, both of which are important concepts in the reactivation of the HCMV or VZV virus in the body.

FIG. 5 is a diagram of the construction of a gene expression cassette for implementation in a HCMV or VZV for delivery into a patient. Shown here is an example of an expression plasmid which may be utilized. Any number of different genes may be selected based on varying treatment plans. A plurality of gene expressions may also be present, rather than a single one, as will be shown in later drawings. Shown is a TERT expression plasmid 540 also known as an expression vector. The main component of such an expression vector is the gene to be transferred into target cells 510. In this example a TERT gene is chosen for patients who may require, for example, increased production of telomerase reverse transcriptase, an important enzyme in the creation of telomerase and the extension of chromosomal telomeres. As disclosed elsewhere herein, other genes may be added or alternatively used.

Extending the telomeres of patient cells is a new concept which may alleviate or prevent certain disorders such as cri du chat or a genetic disorder characterized by a patient's inability to synthesize hTERT due to a chromosomal mutation, and may also prolong the patient's lifespan and/or alter a patient's risk profile for various forms of cancer. A promoter piece of DNA 520 (shown here as an EFla promoter) is included to begin transcription of the target gene in the host cell. Also present is a BGH terminator 530 whose purpose is to terminate the sequence upon transcription, thus providing a TERT cassette in a usable expression plasmid 540. This is a mono-gene cassette, with only one gene inside the plasmid, however it should be obvious to those skilled in the art that multiple genes may be used a multi-gene cassette, and thereby multiple proteins and enzymes may be coded for in the genetic material inserted into a patient, for many potential treatments.

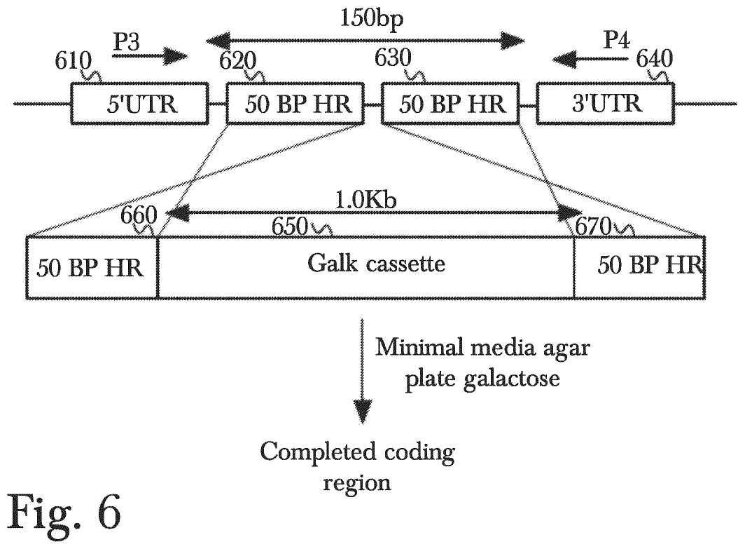

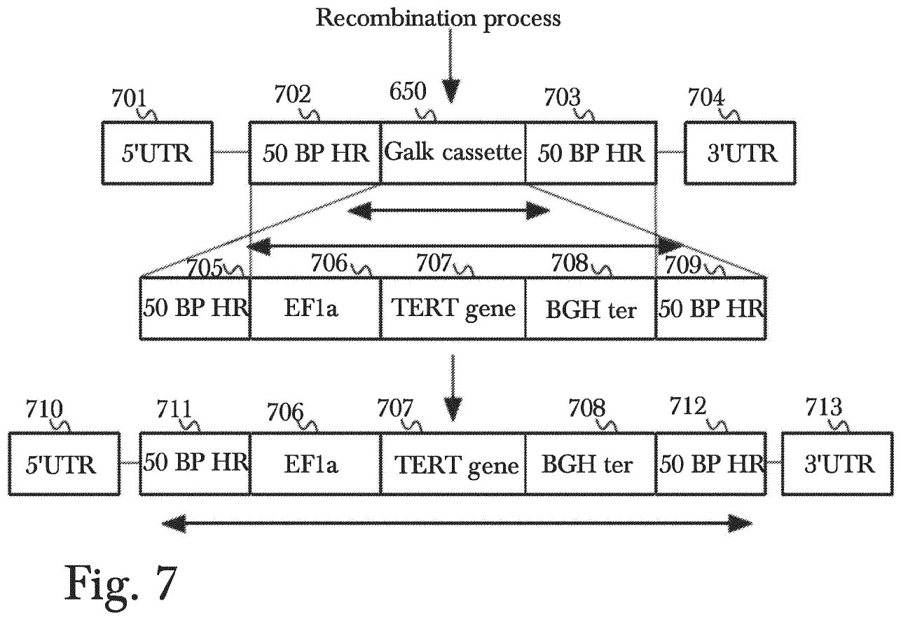

FIG. 6 is a diagram of a partial process of double homologous recombination of genes, according to a preferred aspect. A plurality of 50 base-pair homologous recombination sites 620, 630 exist in a HCMV or VZV BAC, between a 5′ untranslated region 610 and a 3′ untranslated region 640 which mark directional endpoints of an incomplete cassette. Using homologous recombination sites 620, 630, a galK gene cassette 650, which exists as a piece of genetic code between two similar or identical 50 base-pair homologous recombination sites 660, 670, is then recombined and translated into the initial structure containing recombination sites 620, 630 and directional endpoints 610, 640, resulting in a structure as shown in FIG. 7.

FIG. 7 diagrams a partial process of double homologous recombination of genes, according to a preferred aspect. A galK gene cassette 650 is recombined as shown in FIG. 6 and may exist in a HCMV or VZV BAC, now having two 50 base-pair homologous recombination sites 702, 703, and a 5′ UTR 701 leader sequence and 3′ UTR 704 trailer sequence. The entire gene structure a complete coding sequence capable of correctly encoding for desired proteins—in this instance, those coded for by a galK gene, and capable of being injected via a HCMV or VZV or similar virus. A second instance of homologous recombination then takes place with a gene cassette for a TERT gene 707, including an EFla promoter 706 and BGH terminator 708, and two 50 base-pair recombination sites 705, 709.

Recombination with the previous coding sequence results in a complete coding sequence for a TERT gene 707—surrounded by an EFla promoter 706, BGH terminator 708, two 50 base-pair recombination sites 711, 712 and a leading 5′ untranslated region 710 and a trailing 3′ untranslated region 713—ready to administer to a patient via a carrier HCMV or VZV. This general process of multi-step homologous recombination using gene cassettes and BAC's results in a HCMV or VZV BAC ready for insertion into a patient. In other words, a piece of genetic code is compatible and ready for insertion into an HCMV or VZV and to be administered to a patient. Through the process detailed in FIG. 6 and FIG. 7, various genes may be recombined and inserted into cassettes to then be inserted into bacteria and transfected into other organisms. It will be obvious to those with ordinary skill in the art that through the process of double homologous recombination, numerous other genes may be transcribed here for a variety of differing treatments, which may range from treating genetic disorders previously thought to be untreatable, to promoting increased protein production for other treatments, and possibly even cancer treatments for different forms of cancerous tumors by encoding genes which terminate the cancerous cells, depending on what genes are encoded for therapy.

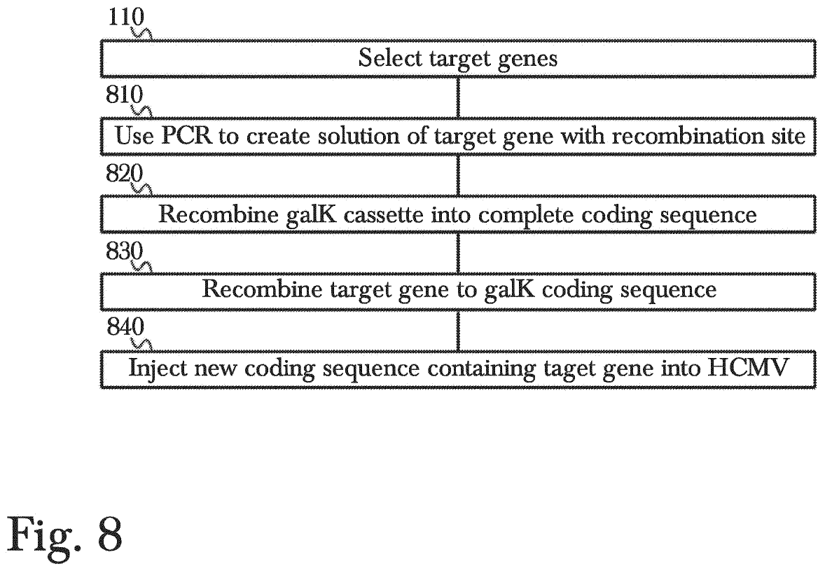

FIG. 8 is a method diagram of gene recombination and construction of desired genes for insertion into a HCMV or VZV. Target genes are selected 110 for human insertion, the selected genes varying depending on the specific medical requirements for a given patient. Gene selection may be based on a desired outcome such as prolonging human life, as with selecting the hTERT gene to eventually be administered to a human patient, or may be based on attempting to cure a genetic disorder, for example cri du chat, Down syndrome, and others, possibly including cancer-causing mutations. Polymerase chain reactions (PCR) are used to create many copies of a desired gene with recombination sites 810 in a solution, as is common in the art, using thermocycling as a key part of the PCR technique for DNA melting and enzyme action to take place. Recombination of, for example, a galK cassette 650 into a complete coding sequence 820 then may take place, using an empty coding sequence which contains homologous recombination sites 620, 630 to write a galK cassette into a coding sequence 820 before a galK cassette is recombined into a target gene 830. An example of a target gene that can be recombined into a coding sequence may be a TERT gene 707, for the purpose of aiding a patient in production of telomerase and extending telomeres at the ends of patient cells, for example. Once a new coding sequence is completed it may be injected into a HCMV or VZV 840 (see FIG. 3) for later administering to a patient intranasally 130, 1020.

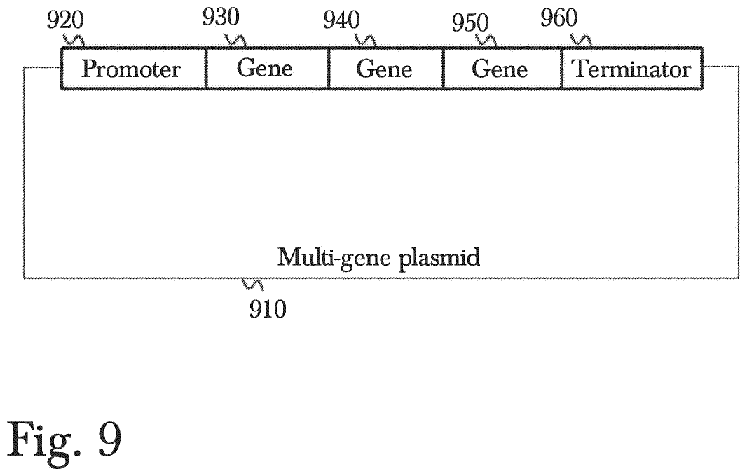

FIG. 9 is a diagram of the construction of a multi-gene cassette with a plurality of genes inserted into a single cassette, for application in a HCMV or VZV, according to a preferred aspect. FIG. 9 shows a similar diagram to that of FIG. 5 but rather than having a single gene plasmid, a possible three-gene plasmid 910 is shown, to illustrate the possibility of a multi-gene plasmid. A promoter 920 exists at one end of the plasmid's genetic code, existing to encourage and start the transcription of the rest of the genetic code of the plasmid, contained in multiple genes 930, 940, 950. A termination string of genetic code 960 exists to stop transcription and signals the end of the genetic code contained in the plasmid. It is possible to have multiple genes, not simply three or one gene, inside a plasmid, which is what this diagram conveys here.

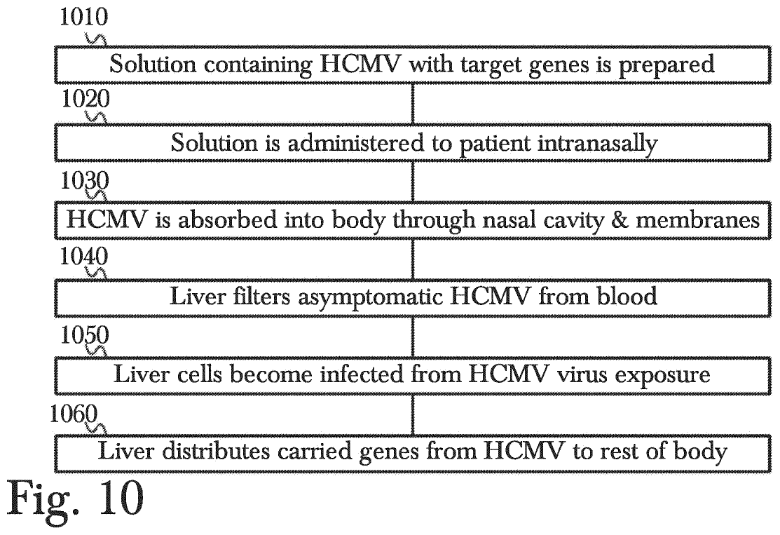

FIG. 10 is a method diagram of HCMV or VZV delivery intranasally and the process of being metabolized into the host body through the liver, according to a preferred embodiment. First, a solution containing HCMV or VZV with desired genetic material must be prepared 1010. This may be accomplished through the processes described earlier including using homologous recombination sites to produce gene cassettes containing desired genetic material (as in FIGS. 6-7), insertion of gene cassettes into viral agents and administering to a patient, or some other method. Target genes may be selected from a pool of potential candidate genes, which may allow for treatment of many different disorders or ailments for a patient, or improve their health even in the absence of a disorder, such as with selecting the hTERT gene to extend patient telomeres and potentially prolong lifespans.

A solution, once prepared 1010, may then be introduced or administered to a patient 1020, including intranasal administration. Intranasal administration of therapeutic solutions provides a cheap and effective way to introduce a gene into an individual's body, allowing it to be absorbed through the nasal cavity and membranes present in the nasal passages of a patient 1030. Once absorbed this way, the viral agent containing the desired gene or genes may then be processed by a patient's liver 1040, which infects liver cells 1050 with the genetic material, and can result in efficient propagation of selected genes through a patient's body 1060, through the bloodstream. In this way, genes may be selected 1010, grown through numerous techniques outlined herein, transfected into a viral agent, administered to a patient 1020, and spread through the patient's body rapidly 1060, resulting in cheap, effective gene therapy for a patient.

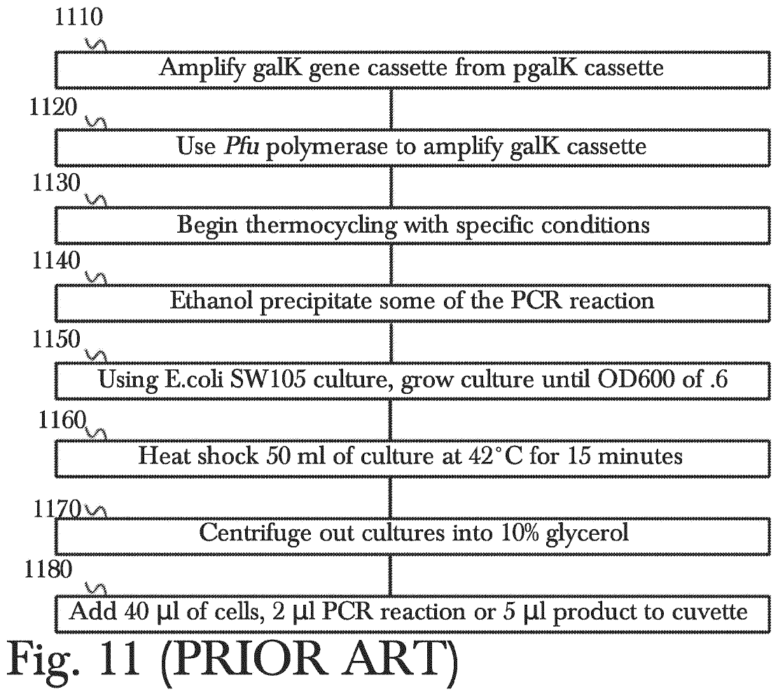

FIG. 11 is a diagram of key steps in a method for bacterial gene recombination to acquire desired genes for later viral transfection. First, a galK gene cassette is amplified from a pgalK template 1110 using appropriately designed primers. Using a polymerase chain reaction (PCR) technique, involving the use of a pfu DNA polymerase enzyme 1120, one may essentially grow many more copies of desired genetic code segments. PCR requires the use of thermocycling 1130 with specific laboratory conditions, which allows for DNA melting and enzyme action to take place, essentially allowing many complex reactions to take place over and over as temperatures are raised and lowered with specific regularity, as is common in the art. Ethanol is used to precipitate a portion of the PCR reactions out 1140, for further use, and for washing.

Using, for example, E. coli SW105 culture, one may then grow a culture of E. coli until OD600 of 0.6 is achieved 1150, indicating a specific desired concentration, before heat shocking 50 ml of the culture at 42° C. for 15 minutes 1160 which induces expression of lambda-Red recombinase genes. One may then centrifuge out cultures into 10% glycerol in two tubes 1170, possibly more than one time and washing in glycerol each time, to achieve a concentration of approximately 200-250 microliters of cells in each tube in 10% glycerol. Electroporation can then be started, firstly by adding 40 μl of cells, 2 20 μl of the PCR reaction or 5 μl of the ethanol-precipitated PCR product, to a cuvette 1180.

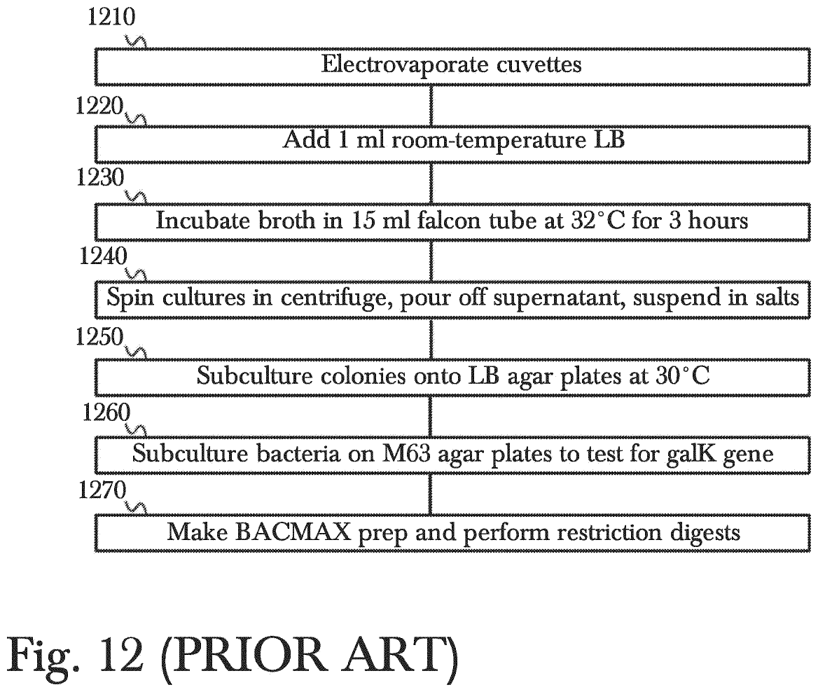

FIG. 12 is a diagram of key steps in a method for bacterial gene recombination to acquire desired genes for later viral transfection. As shown in FIG. 11, once requisite materials and cell are added to a cuvette 1180, electroporation may be conducted 1210, before adding 1 ml of room-temperature lysogeny broth (LB) 1220. This allows cultures to grow in a nutrient rich environment. Incubation of broth can then be performed, in a 15 ml Falcon®tube at 32° C. for 3 hours 1230, with shaking, so as to mix the broth around.

After incubation, cultures are spun at high speed for 5 minutes before supernatant is poured off and the cultures are re-suspended in a solution of 1×M9 salts 1240 comprising 48 mM Na2HPO4, 22 mM KH2PO4, 8.5 mM NaCl, and 18.6 mM NH4Cl. Cultures are then subcultured onto fresh LB agar plates at 30° C. and left to grow overnight 1250, and when they have grown overnight, test that the galK gene makes the cultures sensitive to 2-deoxy-galactose by subculturing them further onto M63 agar plates 1260. Lastly in this process, a lack of unwanted recombination of the BAC in repeat regions is confirmed by making a BACMAX prep and performing multiple restriction digests 1270.

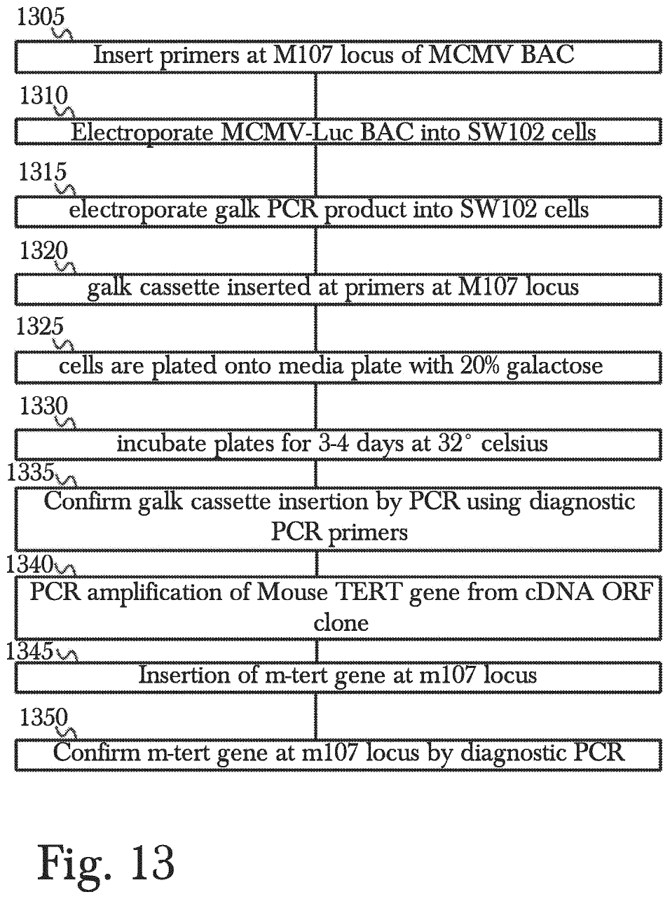

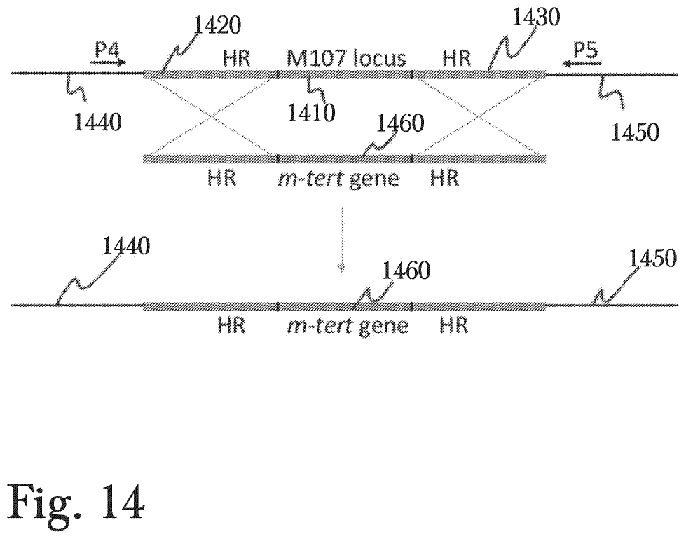

FIG. 13 is a method diagram showing steps in the creation of mouse cytomegalovirus (MCMV)-Luc SW102 electro competent cells, as an alternative method of introducing desired genes into a cytomegalovirus for administration, according to a preferred aspect. Primers are inserted via PCR, at the M107 locus of an MCMV BAC 1305, before the BAC is electroporated into SW102 cells 1310. galK PCR products are then also electroporated into the cells 1315, where a galK cassette is inserted at the location of the primers in the M107 locus 1320. mCMV cells may be used in this methodology as an alternative to HCMV cells. The cells may be placed on a media plate with a 20% galactose solution 1325 for growth of the cells and incubated at 32° C. for three to four days 1330 for optimal growth time. galK cassette insertion by PCR is confirmed using diagnostic PCR primers 1440, 1450, 1335, to ensure successful recombination. The mouse TERT (m-tert) gene may be amplified by PCR techniques from a complimentary DNA (cDNA) open reading frame (ORF) clone 1340, i.e., creating clones of a plasmid containing cDNA for an m-tert gene using PCR. The gene will be recombined at the m107 locus in the MCMV BAC 1345, as shown in FIG. 14, at which point diagnostic primers 1440, 1450 may be used again to confirm the presence of desired genes 1350. Creation of plasmids in this manner may be useful for transfection into other cells including HCMV, among other uses.

FIG. 14 is a diagram showing the insertion of an m-tert gene at a specific locus in a bacterial artificial chromosome in a MCMV, according to a preferred aspect. A M107 locus 1410 in a MCMV is bordered by homologous recombination sites on each side 1420, 1430, which are bordered by diagnostic primers 1440, 1450 respectively, allowing for the recombination of a desired gene cassette into a chromosome. According to a preferred aspect, an m-tert gene cassette 1460 is recombined at the point of the M107 locus 1410 in an MCMV BAC, resulting in the gene cassette 1460 now being opposed by homologous recombination sites 1420, 1430 and diagnostic primers 1440, 1450, at a specific point denoted by the M107 locus 1410 in the bacterial chromosome.

### Example CMV Constructs

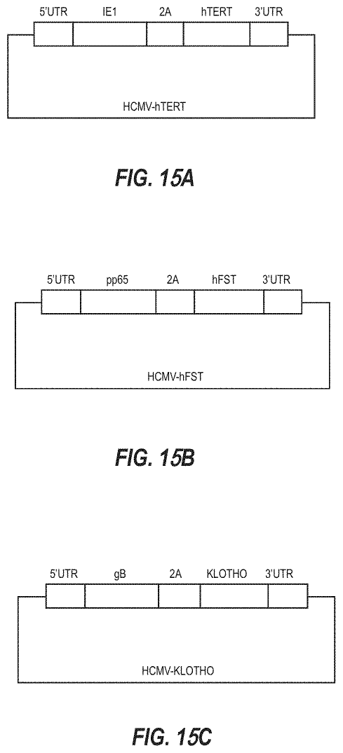

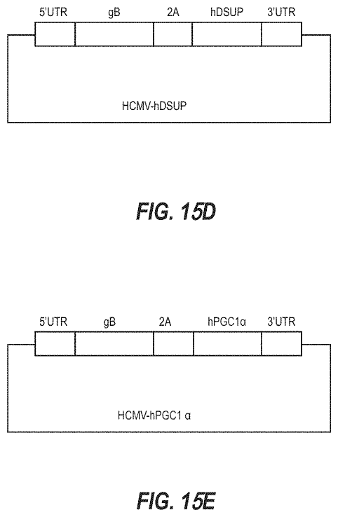

FIGS. 15A-15E illustrate example BAC constructs that may be utilized to generate the CMV vectors disclosed herein. As shown, the constructs include one or more therapeutic genes suitable for administration to human subjects. FIG. 15A shows an example construct that includes hTERT as an exogenous insert. In the illustrated example, the hTERT sequence is fused to the C-terminus (i.e., the portion coding for the C-terminus) of the immediate-early (IE1) gene via a sequence coding for a self-cleaving 2A peptide. The IE1 protein is an early phase protein expressed at high levels during early stages of CMV infection.

FIG. 15B shows an example construct that includes hFST as an exogenous insert. In this example, the hFST sequence is fused to the C-terminus of the pp65 (ppUL83) gene via a sequence coding for a self-cleaving 2A peptide.

FIG. 15C shows an example construct that includes KLOTHO as an exogenous insert. In this example, the KLOTHO sequence is fused to the C-terminus of the gB (envelope glycoprotein B) gene via a sequence coding for a self-cleaving 2A peptide.

FIG. 15D shows an example construct that includes Dsup as an exogenous insert. In this example, the Dsup sequence is fused to the C-terminus of the gB (envelope glycoprotein B) gene via a sequence coding for a self-cleaving 2A peptide.

FIG. 15E shows an example construct that includes PGC1α as an exogenous insert. In this example, the PGC1α sequence is fused to the C-terminus of the gB (envelope glycoprotein B) gene via a sequence coding for a self-cleaving 2A peptide.

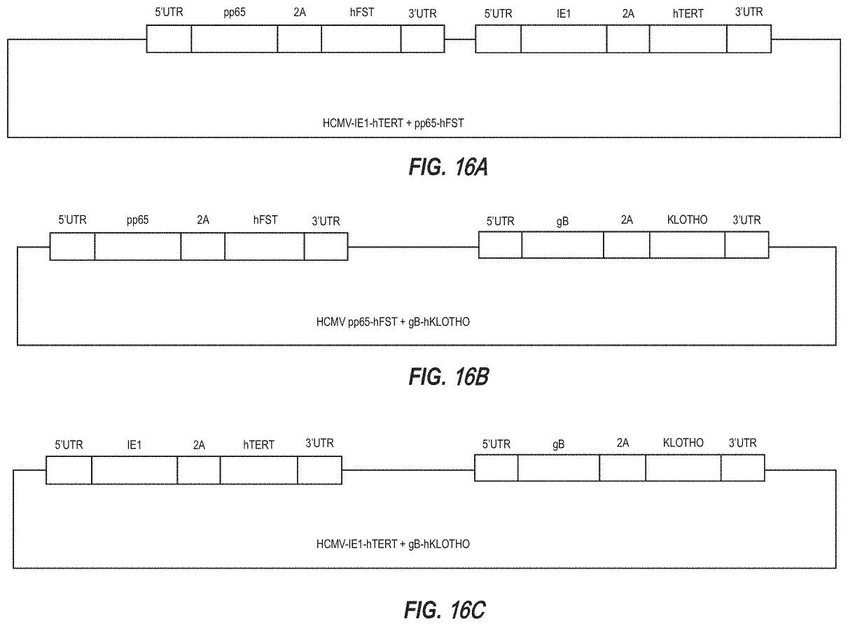

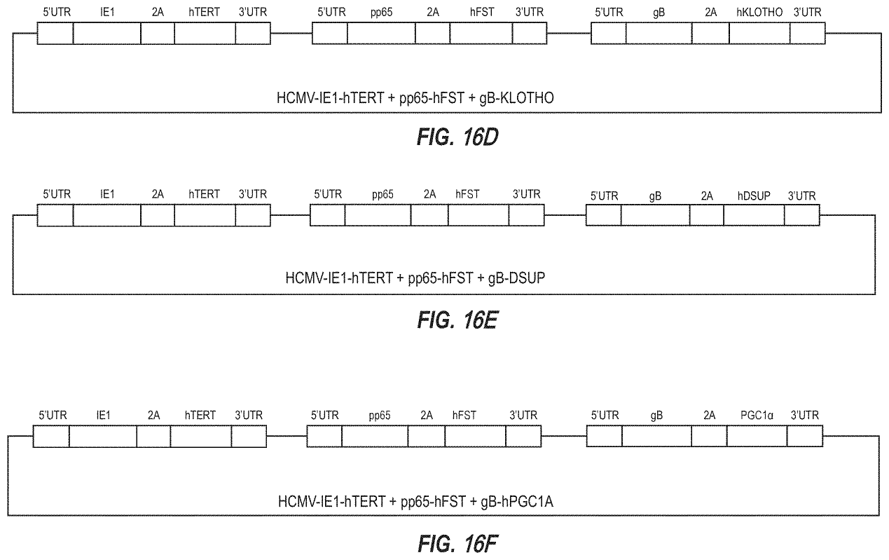

FIGS. 16A-16F illustrate example BAC constructs that each include multiple therapeutic donor genes suitable for administration to human subjects. FIG. 16A shows an example construct that includes both hTERT and hFST as exogenous inserts, wherein the hTERT is joined to the C-terminus of the IE1 gene and the hFST is joined to the C-terminus of the pp65 gene, each via a sequence coding for a self-cleaving 2A peptide.

FIG. 16B shows an example construct that includes both hFST and KLOTHO as exogenous inserts, wherein the hFST is joined to the C-terminus of the pp65 gene and the KLOTHO is joined to the C-terminus of the gB gene, each via a sequence coding for a self-cleaving 2A peptide.

FIG. 16C shows an example construct that includes both hTERT and KLOTHO as exogenous inserts, wherein the hTERT is joined to the C-terminus of the IE1 gene and the KLOTHO is joined to the C-terminus of the gB gene, each via a sequence coding for a self-cleaving 2A peptide.

FIG. 16D shows an example construct that includes hTERT, hFST, and KLOTHO as exogenous inserts, wherein the hTERT is joined to the C-terminus of the IE1 gene, the hFST is joined to the C-terminus of the pp65 gene, and the KLOTHO is jointed to the C-terminus of the gB gene, each via a sequence coding for a self-cleaving 2A peptide.

FIG. 16E shows an example construct that includes hTERT, hFST, and Dsup as exogenous inserts, wherein the hTERT is joined to the C-terminus of the IE1 gene, the hFST is joined to the C-terminus of the pp65 gene, and the Dsup is jointed to the C-terminus of the gB gene, each via a sequence coding for a self-cleaving 2A peptide.

FIG. 16F shows an example construct that includes hTERT, hFST, and PGC1α as exogenous inserts, wherein the hTERT is joined to the C-terminus of the IE1 gene, the hFST is joined to the C-terminus of the pp65 gene, and the PGC1α gene is jointed to the C-terminus of the gB gene, each via a sequence coding for a self-cleaving 2A peptide.

The constructs of this disclosure are not limited to the specific embodiments illustrated in the Figures. For example, other constructs may include one or more donor genes selected from hTERT, hFST, KL, Dsup, PGC1-α, Oct-4, Sox2, KLF4, or any combination thereof. The one or more donor genes may be fused at or near the C-terminus of a CMV gene selected from the IE1 gene, IE2 gene, pp65 gene, UL21.1 gene, UL21.5, gB gene, TRL4 gene, UL89 gene, US3 gene, R160461 gene, R27080 gene, or other suitable CMV gene or reading frame with sufficient expression. Other CMV genes that may be utilized as fusion sites include genes within the gene families RL11, UL14, UL18, UL25, UL82, UL120, US6, US7, US12, and US22. Additional or alternative CMV genes may be utilized as fusion sites for the one or more donor genes. Preferable fusion sites are those that are expressed at relatively high levels to in turn enable high relative expression of the fused gene product. Presently preferred CMV genes to utilized as fusion sites for the one or more donor genes include the IE1 gene, pp65 gene, and gB gene. The one or more donor genes may be fused to their respective CMV genes via a sequence coding for a 2A self-cleaving peptide selected from T2A, P2A, E2A, or F2A, for example.

In some embodiments, a construct includes two donor genes. For example, a construct may include: hTERT and hFST; hTERT and KLOTHO; hTERT and Dsup; hTERT and PGC1-α; hTERT and Oct-4; hTERT and Sox2; hTERT and KLF4; hFST and KLOTHO; hFST and Dsup; hFST and PGC1-α; hFST and Oct-4; hFST and Sox2; hFST and KFL4; KLOTHO and Dsup; KLOTHO and PGC1-α; KLOTHO and Oct-4; KLOTHO and Sox2; KLOTHO and KLF4; Dsup and PGC1-α; Dsup and Oct-4; Dsup and Sox2; Dsup and KLF4; PGC1-α and Oct-4; PGC1-α and Sox2; PGC1-α and KLF4; Oct-4 and Sox2; Oct-4 and KLF4; or Sox2 and KLF4.

In some embodiments, a construct includes three donor genes. For example, a construct may include: hTERT, hFST, and KLOTHO; hTERT, hFST, and Dsup; hTERT, hFST, and PGC1-α; hTERT, hFST, and Oct-4; hTERT, hFST, and Sox2; hTERT, hFST, and KLF4; hTERT, KLOTHO, and Dsup; hTERT, KLOTHO, and PGC1-α; hTERT, KLOTHO, and Oct-4; hTERT, KLOTHO, and Sox2; hTERT, KLOTHO, and KLF4; hTERT, Dsup, and PGC1-α; hTERT, Dsup, and Oct-4; hTERT, Dsup, and Sox2; hTERT, Dsup, and KLF4; hTERT, PGC1-α, and Oct-4; hTERT, PGC1-α, and Sox2; hTERT, PGC1-α, and KLF4; hTERT, Oct-4, and Sox2; hTERT, Oct-4, and KLF4; hTERT, Sox2, and KLF4; hFST, KLOTHO, and Dsup; hFST, KLOTHO, and PGC1-α; hFST, KLOTHO, and Oct-4; hFST, KLOTHO, and Sox2; hFST, KLOTHO, and KLF4; hFST, Dsup, and PGC1-α; hFST, Dsup, and Oct-4; hFST, Dsup, and Sox2; hFST, Dsup, and KLF4; hFST, PGC1-α, and Oct-4; hFST, PGC1-α, and Sox2; hFST, PGC1-α, and KLF4; hFST, Oct-4, and Sox2; hFST, Oct-4, and KLF4; hFST, Sox2, and KLF4; KLOTHO, Dsup, and PGC1-α; KLOTHO, Dsup, and Oct-4; KLOTHO, Dsup, and Sox2; KLOTHO, Dsup, and KLF4; KLOTHO, PGC1-α, and Oct-4; KLOTHO, PGC1-α, and Sox2; KLOTHO, PGC1-α, and KLF4; KLOTHO, Oct-4, and Sox2; KLOTHO, Oct-4, and KLF4; KLOTHO, Sox2, and KLF4; Dsup, PGC1-α, and Oct-4; Dsup, PGC1-α, and Sox2; Dsup, PGC1-α, and KLF4; Dsup, Oct-4, and Sox2; Dsup, Oct-4, and KLF4; Dsup, Sox2, and KLF4; PGC1-α, Oct-4, and Sox2; PGC1-α, Oct-4, and KLF4; PGC1-α, Sox2, and KLF4; or Oct-4, Sox2, and KLF4.

Other embodiments may include more than three donor genes, in any combination. For example, a CMV construct may include more than three of hTERT, hFST, KL, Dsup, PGC1-α, Oct-4, Sox2, and KLF4, in any combination.

In each of the foregoing, the CMV genes utilized as fusion sites may be selected from any of the CMV fusion sites disclosed herein; preferably the fusion sites are selected from the IE1 gene, pp65 gene, and gB gene.

In construct embodiments that include more than one donor gene, each donor gene may be positioned in a separate open reading frame. Alternatively, one or more donor genes may share the same open reading frame. For example, each donor gene may be fused to a separate CMV gene, or alternatively, one or more donor genes may be fused together to form a donor gene fusion product which is then fused to an appropriate CMV gene. Such fused donor genes may be fused (to each other and/or to the CMV gene) via a sequence coding for a 2A self-cleaving peptide, as with other embodiments described herein. Expression of the donor genes may be induced by the same promoter. Alternatively, the donor genes can be independently associated with separate and/or unique promoters that allow individual or separate expression of each donor gene.

Moreover, although BAC constructs represent presently preferred construct embodiments, other construct embodiments may utilize alternative backbones such as P1-derived artificial chromosomes (PACs) and/or suitable plasmids. Where plasmids are utilized, due to the more stringent size constraints, transduction methods may utilize separate, different plasmids that together provide the desired combination of donor genes.

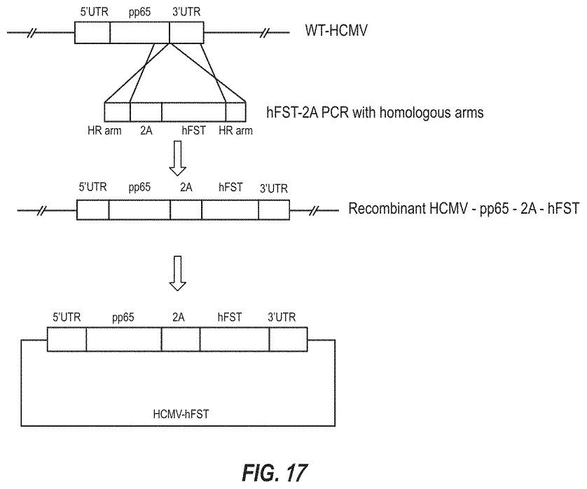

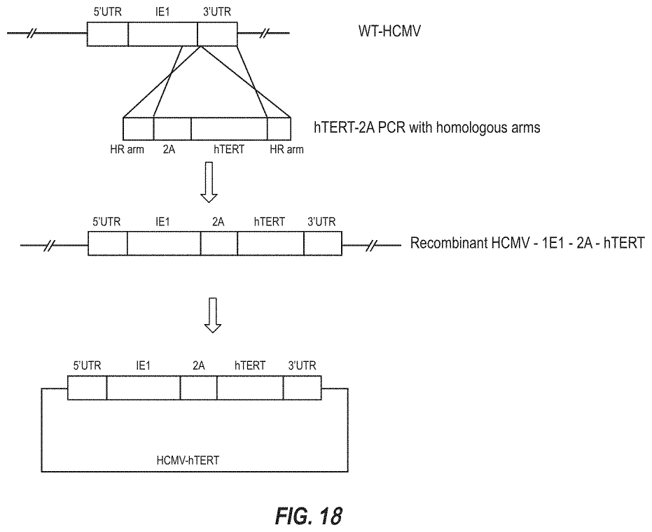

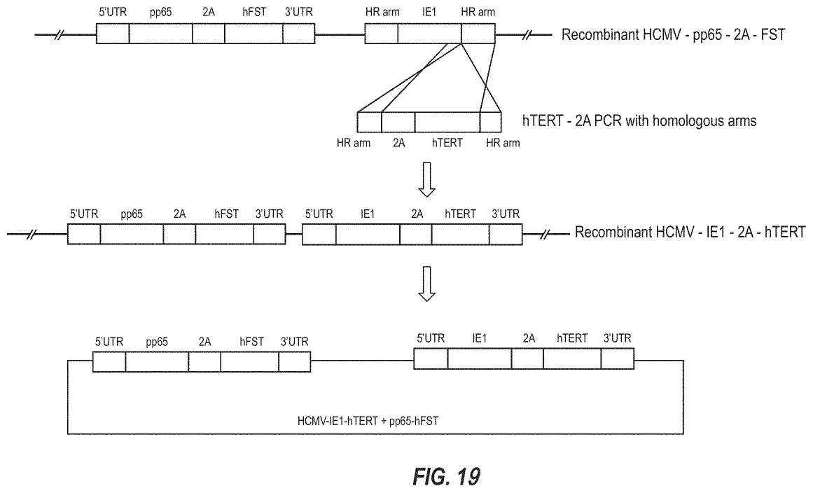

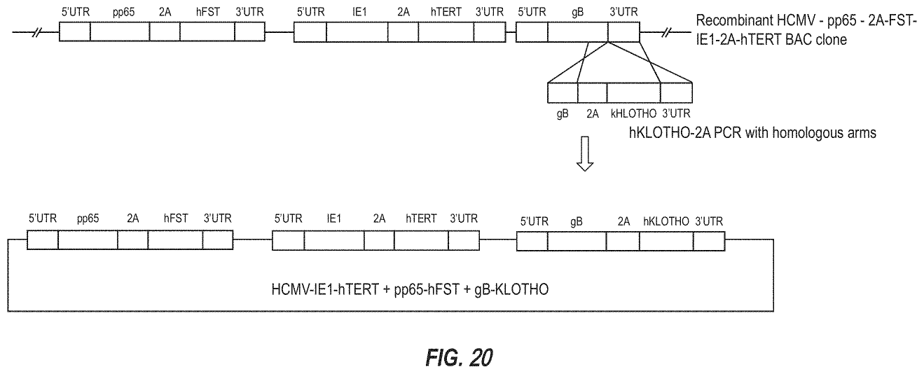

FIGS. 17-20 illustrate methods for manufacturing a CMV-hFST recombinant BAC, CMV-hTERT recombinant BAC, CMV-hFST+hTERT recombinant BAC, and a CMV-hFST+hTERT+KL recombinant BAC, respectively, via homologous recombination. As shown in these figures, the insert is provided with homologous arms (HR arms) on both upstream and downstream ends. The HR arms have substantial identity to the target site in the CMV genome. For example, the upstream HR arm may have substantial identity to a downstream section (i.e., closer to the C-terminus) of the CMV gene targeted for fusion, whereas the downstream HR arm may have substantial identity to the 3′ untranslated region disposed immediately downstream from the C-terminus of the target CMV gene. In this manner, the insert may be inserted into position between the C-terminus of the target CMV gene and the 3′ untranslated region through homologous recombination.

The HR arms may be any length that provides sufficient homologous recombination and integration of the insert into the BAC backbone. In some embodiments, the homologous arms are at least about 25 base pairs in length, or at least about 30 base pairs in length, or at least about 35 base pairs in length, at least about 40 base pairs in length, at least about 45 base pairs in length, and may be up to about 50 base pairs in length, or up to about 55 base pairs in length, or up to about 60 base pairs in length, or up to about 65 base pairs in length, or up to about 70 base pairs in length, or up to about 75 base pairs in length, or up to about 80 base pairs in length, or up to about 90 base pairs in length, or up to about 100 base pairs in length. The HR arms may have a length within a range with endpoints defined by any two of the foregoing values. Effective results have been achieved using HR arms of between about 50 base pairs and about 80 base pairs. Smaller sizes may result in reduced recombination efficiency, while sizes that are too large increase the complexity of the method and are more likely to introduce unwanted side reactions.

### Additional Recombinant Viral Vector Details

The exogenous DNA in the recombinant CMV viruses or vectors described herein can include a promoter. The promoter can be from a herpes virus. For instance, the promoter can be a cytomegalovirus (CMV) promoter, such as a human CMV (HCMV) or murine CMV (MCMV) promoter. The promoter can also be a non-viral promoter such as the EFla promoter. The promoter may be a truncated transcriptionally active promoter which may comprise a region transactivated with a transactivating protein provided by the virus and the minimal promoter region of the full-length promoter from which the truncated transcriptionally active promoter is derived. For purposes of this specification, a “promoter” is composed of an association of DNA sequences corresponding to the minimal promoter and upstream regulatory sequences; a “minimal promoter” is composed of the CAP site plus TATA box (minimum sequences for basic level of transcription; unregulated level of transcription); and “upstream regulatory sequences” are composed of the upstream element(s) and enhancer sequence(s). Further, the term “truncated” indicates that the full-length promoter is not completely present (i.e., that some portion of the full-length promoter has been removed), and the truncated promoter can be derived from a herpesvirus such as MCMV or HCMV (e.g., HCMV-IE or MCMV-IE). Exemplary truncated promoters can be up to a 40% and even up to a 90% reduction in size, from a full-length promoter, based upon base pairs. The promoter can also be a modified non-viral promoter.

Embodiments of the present disclosure also provide an expression cassette for insertion into a recombinant virus or plasmid which may include the truncated transcriptionally active promoter. The expression cassette can further include a functional truncated polyadenylation signal, such as an SV40 polyadenylation signal which is truncated, yet functional. Considering that nature provided a larger signal, it is indeed surprising that a truncated polyadenylation signal is functional; and a truncated polyadenylation signal addresses the insert size limit problems of recombinant viruses such as CMV. The expression cassette can also include exogenous or heterologous DNA with respect to the virus or system into which it is inserted, and that DNA can be exogenous or heterologous DNA as described herein. This DNA can be suitably positioned and operably linked to the promoter for expression. As to HCMV promoters, reference is made to U.S. Pat. Nos. 5,168,062 and 5,385,839, each of which is incorporated herein.

The heterologous or exogenous DNA in exemplary disclosed recombinants preferably encode an expression product, such as a therapeutic gene (e.g., TERT and/or FS344, including human and/or mouse versions thereof, or other version thereof appropriate for the target subject) and/or a fusion protein (e.g., fused with a reporter, such as luciferase, or with an N- or C-terminal epitope tag, such as FLAG, 6X-His, or other epitope tag known to those having skill in the art). With respect to these terms, reference is made to the following discussion, and generally to Kendrew, THE ENCYCLOPEDIA OF MOLECULAR BIOLOGY (Blackwell Science Ltd 1995) and Sambrook, Fritsch, Maniatis, Molecular Cloning, A LABORATORY MANUAL (2d Edition, Cold Spring Harbor Laboratory Press, 1989).

As to size of the DNA incorporated to form recombinant virus/vectors: the skilled artisan can maximize the size of the protein encoded by the DNA sequence to be inserted into the selected viral vector (keeping in mind the packaging limitations of the vector). To minimize the DNA inserted while maximizing or matching the native size of the protein(s) expressed, the DNA sequence(s) can exclude introns (regions of a gene that are transcribed but which are subsequently excised from the primary RNA transcript prior to translation).

With respect to expression of fusion proteins disclosed herein, reference is made to Sambrook, Fritsch, Maniatis, Molecular Cloning, A LABORATORY MANUAL (2d Edition, Cold Spring Harbor Laboratory Press, 1989) (especially Volume 3), and Kendrew, THE ENCYCLOPEDIA OF MOLECULAR BIOLOGY (Blackwell Science Ltd 1995. The teachings of Sambrook et al., can be suitably modified, without undue experimentation, from this disclosure, for the skilled artisan to generate recombinants expressing fusion proteins. With regard to gene therapy, reference is made to U.S. Pat. No. 5,252,479, which is incorporated herein by this reference, together with the documents cited therein and on its face.

It should be understood that the proteins and the nucleic acids encoding them may differ from the exact sequences illustrated and described herein. Thus, the invention contemplates deletions, additions, truncations, and substitutions to the sequences shown, so long as the sequences function in accordance with the methods of the invention. In this regard, substitutions will generally be conservative in nature (i.e., those substitutions that take place within a family of amino acids). For example, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. It is reasonably predictable that an isolated replacement of a leucine with an isoleucine or valine, or vice versa; an aspartate with a glutamate or vice versa; a threonine with a serine or vice versa; or a similar conservative replacement of an amino acid with a structurally related amino acid, will typically not have a major effect on the biological activity. Proteins having substantially the same amino acid sequence as the sequences illustrated and described but possessing minor amino acid substitutions that do not substantially affect the activity or function of the protein are, therefore, within the scope of the invention.

In some embodiments, the therapeutic gene includes a recombinant nucleotide sequence. For example, in one embodiment the nucleotide sequences may be codon optimized, for example the codons may be optimized for human use. As regards codon optimization, the nucleic acid molecules of the invention have a nucleotide sequence that encodes the therapeutic proteins of the invention (i.e., telomerase and/or follistatin) and can be designed to employ codons that are used in the genes of the subject in which the protein is to be produced. Many viruses use a large number of rare codons and, by altering these codons to correspond to codons commonly used in the desired subject, enhanced expression of the recombinant genes can be achieved. In a preferred embodiment, the codons used are “humanized” codons, i.e., the codons are those that appear frequently in highly expressed human genes (see Andre et al., J. Virol. 72:1497-1503, 1998) instead of those codons that are frequently used by or encoded by the vector/virus. Such codon usage provides for efficient expression of the transgenic viral proteins in human cells. Any suitable method of codon optimization may be used. Such methods, and the selection of such methods, are well known to those of skill in the art, and the nucleotide sequences of the inventive vectors described herein can readily be codon optimized in light of the additional teachings provided by this disclosure.

The invention further encompasses nucleotide sequences encoding functionally equivalent variants and derivatives of the CMV vectors disclosed herein. These functionally equivalent variants, derivatives, and fragments display the ability to retain functional activity. For instance, changes in a DNA sequence that do not change the encoded amino acid sequence, as well as those that result in conservative substitutions of amino acid residues, one or a few amino acid deletions or additions, and substitution of amino acid residues by amino acid analogs are those which will not significantly affect properties of the encoded polypeptide. Conservative amino acid substitutions include glycine/alanine; valine/isoleucine/leucine; asparagine/glutamine; aspartic acid/glutamic acid; serine/threonine/methionine; lysine/arginine; and phenylalanine/tyrosine/tryptophan. In some embodiments, the nucleotides have at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% homology or identity to the natural form of the polypeptide of interest.

For the purposes of the present invention, sequence identity or homology is determined by comparing the sequences when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin & Altschul, Proc. Natl. Acad. Sci. USA 1990; 87: 2264-2268, modified as in Karlin & Altschul, Proc. Natl. Acad. Sci. USA 1993; 90: 5873-5877. Another example of a mathematical algorithm used for comparison of sequences is the algorithm of Myers & Miller, CABIOS 1988; 4: 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988; 85: 2444-2448. Advantageous for use according to the present invention is the WU-BLAST (Washington University BLAST) version 2.0 software. WU-BLAST version 2.0 executable programs for several UNIX platforms can be downloaded from ftp://blast.wustl.edu/blast/executables. This program is based on WU-BLAST version 1.4, which in turn is based on the public domain NCBI-BLAST version 1.4 (Altschul & Gish, 1996, Local alignment statistics, Doolittle ed., Methods in Enzymology 266: 460-480; Altschul et al., Journal of Molecular Biology 1990; 215: 403-410; Gish & States, 1993; Nature Genetics 3: 266-272; Karlin & Altschul, 1993; Proc. Natl. Acad. Sci. USA 90: 5873-5877).

The various recombinant nucleotide sequences and recombinant vectors can be made using standard recombinant DNA and cloning techniques, such as those disclosed in “Molecular Cloning: A Laboratory Manual”, second edition (Sambrook et al. 1989). However, it should generally be appreciated that the vectors used in accordance with the embodiments of the present disclosure are typically chosen such that they contain a suitable gene regulatory region, such as a promoter or enhancer, to allow for the desired level of expression of the encoded therapeutic gene. Thus, one skilled in the art can create recombinants expressing a therapeutic gene of interest and use the recombinants from this disclosure and the knowledge in the art, without undue experimentation. Moreover, from the disclosure herein and the knowledge in the art, no undue experimentation is required for the skilled artisan to construct a recombinant virus/vector that expresses a therapeutic gene of interest or for the skilled artisan to use such a recombinant virus/vector.

Embodiments of the present disclosure enable various treatment methods. For example, a treatment method can include a longitudinal treatment method where a plurality of therapeutically effective dosages are provided to a patient over a period of time. In some instances, the period of time is as long as 6-12 months or more, with dosages being administered to the patient annually, semi-annually, every other month, every month, every three weeks, every other week, every week, or more regularly, for example. In an exemplary treatment method, a therapeutically effective dosage is administered to the patient every month for 8 months. In some embodiments, the patient is a middle-aged or elderly patient. For example, the patient can be a human patient that is 30 years or older, 35 years or older, 40 years or older, 45 years or older, 50 years or older, 55 years or older, 60 years or older, 65 years or older, 70 years or older, or older (or can be any age falling within a range formed by the foregoing ages). The patient can also be an animal patient having an analogously middle-age or elderly age.

### Pharmaceutical Compositions

While it is possible for the compounds described herein to be administered alone, it may be preferable to formulate the compounds as pharmaceutical compositions (e.g., formulations). As such, in yet another aspect, pharmaceutical compositions useful in the methods and uses of the disclosed embodiments are provided. A pharmaceutical composition is any composition that may be administered in vitro or in vivo or both to a subject to treat, prevent, or ameliorate a condition or may otherwise be administered prophylactically to improve or maintain the health of the subject. In a preferred embodiment, a pharmaceutical composition may be administered in vivo. A subject may include one or more cells or tissues, or organisms. In some exemplary embodiments, the subject is an animal. In some embodiments, the animal is a mammal. The mammal may be a human, mouse, or primate in some embodiments. A mammal includes any mammal, such as by way of non-limiting example, cattle, pigs, sheep, goats, horses, camels, buffalo, primates, cats, dogs, rats, mice, and humans.

The compositions of the invention may be injectable suspensions, solutions, sprays, lyophilized powders, syrups, elixirs, and the like. Any suitable form of composition may be used. To prepare such a composition, a nucleic acid or vector of the invention, having the desired degree of purity, is mixed with one or more pharmaceutically acceptable carriers and/or excipients.

As used herein the terms “pharmaceutically acceptable” and “physiologically acceptable” mean a biologically compatible formulation, gaseous, liquid, or solid, or mixture thereof, which is suitable for one or more routes of administration, in vivo delivery, or contact. A formulation is compatible in that it does not destroy activity of the engineered viral vector or proteins made thereby or induce adverse side effects that outweigh any prophylactic or therapeutic effect or benefit.

In an embodiment, the pharmaceutical compositions may be formulated with pharmaceutically acceptable excipients such as carriers, solvents, stabilizers, adjuvants, diluents, etc., depending upon the particular mode of administration and dosage form. The pharmaceutical compositions should generally be formulated to achieve a physiologically compatible pH and may range from a pH of about 3 to a pH of about 11, preferably about pH 3 to about pH 7, depending on the formulation and route of administration. In alternative embodiments, it may be preferred that the pH is adjusted to a range from about pH 5 to about pH 8. More particularly, the pharmaceutical compositions may comprise a therapeutically or prophylactically effective amount of at least one compound as described herein, together with one or more pharmaceutically acceptable excipients. Optionally, the pharmaceutical compositions may comprise a combination of the compounds described herein or may include a second recombinant vector, or second protein encoded thereby, that is useful in the treatment or prevention of aging or aging-related phenomena, as discussed herein.

Formulations, for example, for parenteral or oral administration, are most typically solids, liquid solutions, emulsions, or suspensions, while inhalable formulations for intranasal or pulmonary administration are generally liquids or powders. An exemplary pharmaceutical composition may be formulated as a lyophilized solid that is reconstituted with a physiologically compatible solvent or carrier prior to administration. Other suitable carriers or diluents can be water or a buffered saline, with or without a preservative. The recombinant vector may be lyophilized for resuspension at the time of administration or can be in solution.

Acceptable carriers, excipients, or stabilizers are nontoxic to recipients at the dosages and concentrations employed, and include, but are not limited to, water, saline, phosphate buffered saline, dextrose, glycerol, ethanol, or combinations thereof, buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN® PLURONICS® or polyethylene glycol (PEG).

Pharmaceutically acceptable excipients are determined in part by the particular composition being administered, as well as by the particular method used to administer the composition. Accordingly, there exists a wide variety of suitable formulations of pharmaceutical compositions (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants such as ascorbic acid; chelating agents such as EDTA; carbohydrates such as dextrin, hydroxyalkylcellulose, hydroxyalkylmethylcellulose, stearic acid; liquids such as oils, water, saline, glycerol, and ethanol; wetting or emulsifying agents; pH buffering substances; and the like. Liposomes are also included within the definition of pharmaceutically acceptable excipients.

For example, pharmaceutically acceptable excipients particularly suitable for use in conjunction with tablets include, for example, inert diluents, such as celluloses, calcium or sodium carbonate, lactose, calcium, or sodium phosphate; disintegrating agents, such as cross-linked povidone, maize starch, or alginic acid; binding agents, such as povidone, starch, gelatin, or acacia; and lubricating agents, such as magnesium stearate, stearic acid, or talc. Pharmaceutical compositions may also be formulated as dispersible powders and granules suitable for preparation of a suspension by the addition of suitable excipients.

As another example, pharmaceutical compositions may be formulated as suspensions comprising a recombinant vector/virus disclosed herein in admixture with at least one pharmaceutically acceptable excipient suitable for the manufacture of a suspension. Excipients suitable for use in connection with suspensions include suspending agents, such as sodium carboxymethylcellulose, methylcellulose, hydroxypropyl methylcellulose, sodium alginate, polyvinylpyrrolidone, gum tragacanth, gum acacia, dispersing, or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate); polysaccharides and polysaccharide-like compounds (e.g., dextran sulfate); glycoaminoglycans and glycosaminoglycan-like compounds (e.g., hyaluronic acid); and thickening agents, such as carbomer, beeswax, hard paraffin, or cetyl alcohol. The suspensions may also contain one or more preservatives such as acetic acid, methyl and/or n-propyl p-hydroxy-benzoate; one or more coloring agents; one or more flavoring agents; and one or more sweetening agents such as sucrose or saccharin.

The immunogenic compositions can be designed to introduce the viral vectors to a desired site of action and release it at an appropriate and controllable rate. For example, controlled release preparations can be produced by the use of polymers to complex or absorb the immunogen and/or immunogenic composition. A controlled-release formulation can be prepared using appropriate macromolecules (for example, polyesters, polyamino acids, polyvinyl, pyrrolidone, ethylenevinylacetate, methylcellulose, carboxymethylcellulose, or protamine sulfate) known to provide the desired controlled release characteristics or release profile. Another possible method to control the duration of action by a controlled-release preparation is to incorporate the active ingredients into particles of a polymeric material such as, for example, polyesters, polyamino acids, hydrogels, polylactic acid, polyglycolic acid, copolymers of these acids, or ethylene vinylacetate copolymers.

Alternatively, instead of incorporating these active ingredients into polymeric particles, it is possible to entrap these materials into microcapsules. Microencapsulation has been applied to the injection of microencapsulated pharmaceuticals to give a controlled release. A number of factors contribute to the selection of a particular polymer for microencapsulation. The reproducibility of polymer synthesis and the microencapsulation process, the cost of the microencapsulation materials and process, the toxicological profile, the requirements for variable release kinetics and the physicochemical compatibility of the polymer and the viral vectors are all factors that must be considered. Examples of useful polymers are polycarbonates, polyesters, polyurethanes, polyorthoesters, and polyamides, particularly those that are biodegradable.

Microcapsules can be prepared, for example, by coacervation techniques or by interfacial polymerization, for example, hydroxymethylcellulose or gelatin-microcapsule and poly-(methylmethacrylate) microcapsule, respectively, in colloidal drug delivery systems (for example, liposomes, albumin microspheres, microemulsions, nanoparticles and nanocapsules) or in macroemulsions. Such techniques are disclosed in New Trends and Developments in Vaccines, Voller et al. (eds.), University Park Press, Baltimore, Md., 1978 and Remington's Pharmaceutical Sciences, 16th edition.

A frequent choice of a carrier for pharmaceuticals is poly (d,1-lactide-co-glycolide) (PLGA). This is a biodegradable polyester that has a long history of medical use in erodible sutures, bone plates, and other temporary prostheses where it has not exhibited any toxicity. A wide variety of pharmaceuticals, including peptides and antigens, have been formulated into PLGA microcapsules. A body of data has accumulated on the adaption of PLGA for the controlled release of compounds, for example, as reviewed by Eldridge, J. H., et al., Current Topics in Microbiology and Immunology. 1989, 146:59-66. The entrapment of compounds in PLGA microspheres of 1 to 10 microns in diameter has been shown to have a remarkable adjuvant effect when administered orally. The PLGA microencapsulation process uses a phase separation of a water-in-oil emulsion. The compound of interest is prepared as an aqueous solution and the PLGA is dissolved in suitable organic solvents such as methylene chloride and ethyl acetate. These two immiscible solutions are co-emulsified by high-speed stirring. A non-solvent for the polymer is then added, causing precipitation of the polymer around the aqueous droplets to form embryonic microcapsules. The microcapsules are collected and stabilized with one of an assortment of agents (polyvinyl alcohol (PVA), gelatin, alginates, polyvinylpyrrolidone (PVP), methyl cellulose), and the solvent removed by either drying in vacuo or by solvent extraction.

The pharmaceutical compositions may also be in the form of oil-in water emulsions. The oil-in-water emulsion can be based, for example, on light liquid paraffin oil (European Pharmacopea type); isoprenoid oil such as squalane, squalene, EICOSANE™ or tetratetracontane; oil resulting from the oligomerization of alkene(s), e.g., isobutene or decene; esters of acids or of alcohols containing a linear alkyl group, such as plant oils, ethyl oleate, propylene glycol di(caprylate/caprate), glyceryl tri(caprylate/caprate) or propylene glycol dioleate; esters of branched fatty acids or alcohols, e.g., isostearic acid esters. The oil advantageously is used in combination with emulsifiers to form the emulsion. The emulsifiers can be nonionic surfactants, such as esters of sorbitan, mannide (e.g., anhydromannitol oleate), glycerol, polyglycerol, propylene glycol, and oleic, isostearic, ricinoleic, or hydroxystearic acid, which are optionally ethoxylated, and polyoxypropylene-polyoxyethylene copolymer blocks, such as the Pluronic® products, e.g., L121. The adjuvant can be a mixture of emulsifier(s), micelle-forming agent, and oil such as that which is commercially available under the name Provax® (IDEC Pharmaceuticals, San Diego, Calif.). The emulsion may also contain sweetening and flavoring agents. Syrups and elixirs may be formulated with sweetening agents, such as glycerol, sorbitol, or sucrose. Such formulations may also contain a demulcent, a preservative, a flavoring, or a coloring agent.

Additionally, the pharmaceutical compositions may be in the form of a sterile injectable preparation, such as a sterile injectable aqueous emulsion or oleaginous suspension. This emulsion or suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,2-propane-diol.

The sterile injectable preparation may also be prepared as a lyophilized powder. Among the acceptable vehicles and solvents that may be employed are water, Ringer's solution, and an isotonic sodium chloride solution. In addition, sterile fixed oils may be employed as a solvent or suspending medium. For this purpose, any bland fixed oil may be employed including synthetic mono- or diglycerides. In addition, fatty acids such as oleic acid may be used in the preparation of injectables.

In some embodiments, cyclodextrins may be added as aqueous solubility enhancers. Preferred cyclodextrins include hydroxypropyl, hydroxyethyl, glucosyl, maltosyl and maltotriosyl derivatives of α-, β-, and γ-cyclodextrin. An exemplary cyclodextrin solubility enhancer is hydroxypropyl-o-cyclodextrin (BPBC), which may be added to any of the above-described compositions to further improve the aqueous solubility characteristics of the compounds of the embodiments. In one embodiment, the composition comprises about 0.1% to about 20% hydroxypropyl-o-cyclodextrin, more preferably about 1% to about 15% hydroxypropyl-o-cyclodextrin, and even more preferably from about 2.5% to about 10% hydroxypropyl-o-cyclodextrin. The amount of solubility enhancer employed will depend on the amount of the compound of the embodiments in the composition.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, but are not limited to, mineral salts (e.g., AlK(SO₄)₂, AlNa(SO₄)₂, AlNH(SO₄)₂, silica, alum, Al(OH)₃, Ca₃(PO₄)₂, kaolin, or carbon), polynucleotides with or without immune stimulating complexes (ISCOMs) (e.g., CpG oligonucleotides, such as those described in Chuang, T. H. et al., (2002) J. Leuk. Biol. 71(3): 538-44; Ahmad-Nejad, P. et al. (2002) Eur. J. Immunol. 32(7): 1958-68; poly IC or poly AU acids, polyarginine with or without CpG (also known in the art as IC31; see Schellack, C. et al. (2003) Proceedings of the 34th Annual Meeting of the German Society of Immunology; Lingnau, K. et al. (2002) Vaccine 20(29-30): 3498-508), JuvaVax (U.S. Pat. No. 6,693,086), certain natural substances (e.g., wax D-form Mycobacterium tuberculosis, substances found in Cornyebacterium parvum, Bordetella pertussis, or members of the genus Brucella), flagellin (Toll-like receptor 5 ligand; see McSorley, S. J. et al. (2002) J. Immunol. 169(7): 3914-9), saponins such as Q521, Q517, and QS7 (U.S. Pat. Nos. 5,057,540; 5,650,398; 6,524,584; 6,645,495), monophosphoryl lipid A, in particular, 3-de-O-acylated monophosphoryl lipid A (3D-MPL), imiquimod (also known in the art as IQM and commercially available as Aldara®); U.S. Pat. Nos. 4,689,338; 5,238,944; Zuber, A. K. et al. (2004) 22(13-14): 1791-8), and the CCRS inhibitor CMPD167 (see Veazey, R. S. et al. (2003) J. Exp. Med. 198: 1551-1562). Aluminum hydroxide or phosphate(alum) are commonly used at 0.05 to 0.1% solution in phosphate buffered saline. Other adjuvants that can be used include cholera toxin, especially CTA1-DD/ISCOMs (see Mowat, A. M. et al. (2001) J. Immunol. 167(6): 3398-405); polyphosphazenes (Allcock, H. R. (1998) App. Organometallic Chem. 12(10-11): 659-666; Payne, L. G. et al. (1995) Pharm. Biotechnol. 6: 473-93); cytokines such as, but not limited to, IL-2, IL-4, GM-CSF, IL-12, IL-15 IGF-1, IFN-α, IFN-β, and IFN-γ (Boyer et al., (2002) J. Liposome Res. 121:137-142; WO01/095919); immunoregulatory proteins such as CD4OL (ADX40; see, for example, WO03/063899), and the CD1a ligand of natural killer cells (also known as CRONY or a-galactosyl ceramide; see Green, T. D. et al., (2003) J. Virol. 77(3): 2046-2055); immunostimulatory fusion proteins such as IL-2 fused to the Fc fragment of immunoglobulins (Barouch et al., Science 290:486-492, 2000); and co-stimulatory molecules B7.1 and B7.2 (Boyer)—all of which can be administered either as proteins or in the form of DNA, in the same viral vectors as those encoding the therapeutic gene(s) of the embodiments disclosed herein or on separate expression vectors.

### Dosages and Treatment Regimens

Pharmaceutical compositions disclosed herein contain a total amount of the active ingredient(s) sufficient to achieve an intended therapeutic effect. The pharmaceutical compositions may, for convenience, be prepared or provided as a unit dosage form. Preparation techniques include bringing into association the active ingredient (e.g., the recombinant virus/vectors) and pharmaceutical carrier(s) and/or excipient(s). In general, pharmaceutical compositions are prepared by uniformly and intimately associating the active ingredient with liquid carriers or finely divided solid carriers or both, and then, if necessary, shaping the product. For example, a tablet may be made by compression or molding. Compressed tablets may be prepared by compressing, in a suitable machine, an active ingredient in a free-flowing form such as a powder or granules, optionally mixed with a binder, lubricant, inert diluent, preservative, surface-active or dispersing agent. Molded tablets may be produced by molding, in a suitable apparatus, a mixture of powdered compound moistened with an inert liquid diluent. The tablets may optionally be coated or scored and may be formulated so as to provide a slow or controlled release of the active ingredient therein.

Compounds including disclosed pharmaceutical compositions can be packaged in unit dosage forms for ease of administration and uniformity of dosage. A “unit dosage form” as used herein refers to a physically discrete unit suited as unitary dosages for the subject to be treated; each unit containing a predetermined quantity of compound optionally in association with a pharmaceutical carrier (e.g., excipient, diluent, vehicle or filling agent) which, when administered in one or more doses, is calculated to produce a desired effect (e.g., prophylactic or therapeutic effect or benefit). Unit dosage forms can contain a weekly or monthly dose, or an appropriate fraction thereof, of an administered compound. Unit dosage forms also include, for example, capsules, troches, cachets, lozenges, tablets, ampules, and vials, which may include a composition in a freeze-dried or lyophilized state; a sterile liquid carrier, for example, can be added prior to administration or delivery in vivo. Unit dosage forms additionally include, for example, ampules and vials with liquid compositions disposed therein. The individual unit dosage forms can be included in multi-dose kits or containers. Pharmaceutical formulations can be packaged in single or multiple unit dosage forms for ease of administration and uniformity of dosage.

The compositions disclosed herein can be administered in accordance with the methods at any frequency and as a single bolus or multiple doses, for as long as appropriate. Exemplary frequencies are typically from 1-5 times, 1-3 times, 2-times, or once monthly. Timing of contact and administration ex vivo or in vivo can be dictated by the infection or pathogenesis of the viral vector used or by the concentration of the therapeutic protein in the patient (e.g., in serum or within specified organ tissue). In some embodiments, the compounds will be administered for a period of continuous therapy, for example for a week or more, or for months or years. Long-acting pharmaceutical compositions may be administered twice a week, every 3 to 4 days, every week, or once a month depending on half-life and clearance rate of the particular formulation. For example, in an embodiment, a pharmaceutical composition contains an amount of a compound as described herein that is selected for administration to a patient once a month for 6-12 months.

Doses may vary depending upon whether the treatment is therapeutic or prophylactic, the onset, progression, severity, frequency, duration, probability of or susceptibility of the age-related symptom, the type pathogenesis to which treatment is directed, clinical endpoint desired, previous, simultaneous or subsequent treatments, general health, age, gender or race of the subject, bioavailability, potential adverse systemic, regional or local side effects, the presence of other disorders or diseases in the subject, and other factors that will be appreciated by the skilled artisan (e.g., medical or familial history). Dose amount, frequency or duration may be increased or reduced, as indicated by the clinical outcome desired, status of the symptom(s) or pathology, and any adverse side effects of the treatment or therapy. The skilled artisan will appreciate the factors that may influence the dosage, frequency, and timing required to provide an amount sufficient or effective for providing a prophylactic or therapeutic effect or benefit. The exact dosage will be determined by the practitioner, in light of factors related to the subject that requires treatment. Dosage and administration are adjusted to provide sufficient levels of the active agent(s) or to maintain the desired effect.

The dosage may range broadly, depending upon the desired effects and the therapeutic indication. Alternatively, dosages may be based and calculated upon the per unit weight of the patient, as understood by those of skill in the art. In instances where human dosages for compounds have been established for at least some condition, those same dosages may be used, or dosages that are between about 0.1% and 500%, more preferably between about 25% and 250% of the established human dosage. Where no human dosage is established, as will be the case for newly discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

Dosage amount and interval may be adjusted individually to provide plasma levels of the active protein, which are sufficient to maintain the modulating effects, or minimal effective concentration (MEC) thereof. For example, therapeutic dosages of follistatin may result in plasma levels of 0.05 mg/mL, 0.1 mg/mL, 0.5 mg/mL, 1 mg/mL, 5 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, a range bounded by any two of the aforementioned numbers, or about any of the aforementioned numbers and ranges. As another example, therapeutic dosages of telomerase may result in plasma levels of 100 mg/mL, 150 mg/mL, 200 mg/mL, 250 mg/mL, 300 mg/mL, 350 mg/mL, 400 mg/mL, 450 mg/mL, 500 mg/mL, 550 mg/mL, 600 mg/mL, a range bounded by any two of the aforementioned numbers, or about any of the aforementioned numbers and ranges. In some embodiments, the therapeutic dose is sufficient to establish plasma levels in the range of about 250 mg/mL to about 400 mg/mL. The MEC may vary for each compound but can be estimated from in vitro or ex vivo data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration. It should be appreciated that the desired serum concentration of target protein may be adjusted or dosed according to the total number of viral genomes per kg of patient body weight required to reach the desired serum concentration.

As described herein, the methods of the embodiments also include the use of a compound or compounds as described herein together with one or more additional therapeutic agents for the treatment of aging-related disorders or conditions. Thus, for example, the disclosed vectors may be: (1) co-formulated and administered or delivered simultaneously in a combined formulation; (2) delivered by alternation or in parallel as separate formulations; or (3) by any other combination therapy regimen known in the art. When delivered in alternation therapy, the methods described herein may comprise administering or delivering the active ingredients sequentially (e.g., in separate solution, emulsion, suspension, tablets, pills or capsules) or by different injections in separate syringes. In general, during alternation therapy, an effective dosage of each active ingredient is administered sequentially (e.g., serially), whereas in simultaneous therapy, effective dosages of two or more active ingredients are administered together. Various sequences of intermittent combination therapy may also be used.

As used herein, all numerical values or numerical ranges include integers within such ranges and fractions of the values or the integers within ranges unless the context clearly indicates otherwise. Thus, to illustrate, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

As used herein, “co-administration” means concurrently or administering one substance followed by beginning the administration of a second substance within 24 hours, 20 hours, 16 hours, 12 hours, 8 hours, 4 hours, 1 hour, 30 minutes, 15 minutes, 5 minutes, 1 minute, a range bounded by any two of the aforementioned numbers, and/or about any of the aforementioned numbers. In some embodiments, co-administration is concurrent.

Some embodiments are directed to the use of companion diagnostics to identify an appropriate treatment for the patient. A companion diagnostic is an in vitro diagnostic test or device that provides information that is highly beneficial, or in some instances essential, for the safe and effective use of a corresponding therapeutic composition. Such tests or devices can identify patients likely to be at risk for adverse reactions as a result of treatment with a particular therapeutic composition. Such tests or devices can also monitor responsiveness to treatment (or estimate responsiveness to possible treatments). Such monitoring may include schedule, dose, discontinuation, or combinations of therapeutic compositions. In some embodiments, the therapeutic gene is selected by measuring a biomarker in the patient. The term biomarker includes, but is not limited to, genetic elements (e.g., expression level of a genetic element) or proteins (e.g., increase/decrease in expression level of a protein or concentration within a specific tissue or organ).

### Examples

### Example 1: Cells, Media, and Viruses

An MCMV bacterial artificial chromosome (MCMV-BAC) Smith strain was used. Mouse fibroblast 3T3 cells were used for the MCMV culture and growth assays and were cultured in minimal essential media (MEM) with 10% fetal bovine serum (FBS), 1% penicillin and streptomycin (P/S) at 37° C., 5% CO2. Approved Institutional Biosafety Committee (IBC) and IACUC protocol were followed.

### Example 2: Construction and Characterization of Recombinant MCMV_{Luc-TERT} and MCMV_{Luc-FS344}

Recently, the CMV vector has emerged as a potential delivery vector for expressing therapeutic molecules, including proteins. CMV can infect different cell types in the body and is thus able to deliver the target proteins to numerous cell types. More specifically, CMV can infect fibroblast, hepatocytes, endothelial cells, macrophages, epithelial cells, lymphocytes, retinal pigment epithelial cells, and cells of the gastrointestinal tract. Therefore, CMV can infect and deliver its target antigens to different cell types in various organs of the body. Moreover, CMV can overcome the pre-existing immunity to efficiently express the protein of interest. Furthermore, CMV has a large genome size owing to its tremendous capacity to incorporate multiple genes and express them simultaneously.

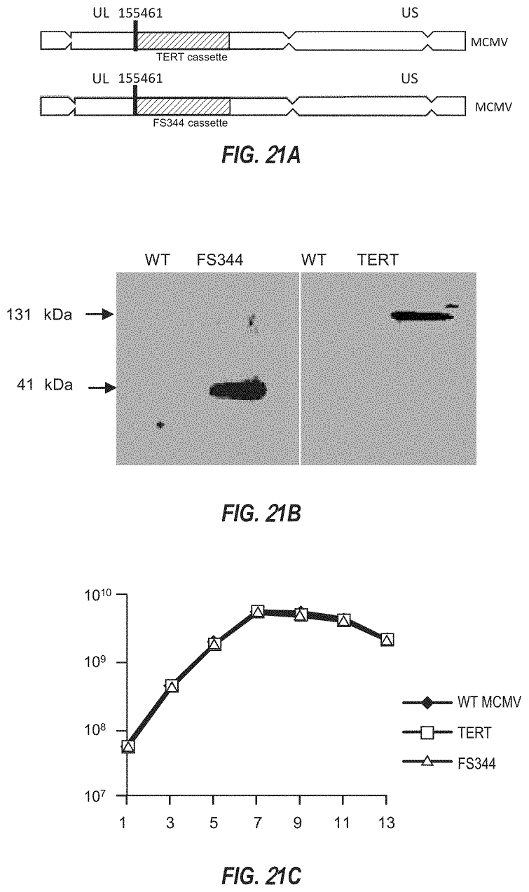

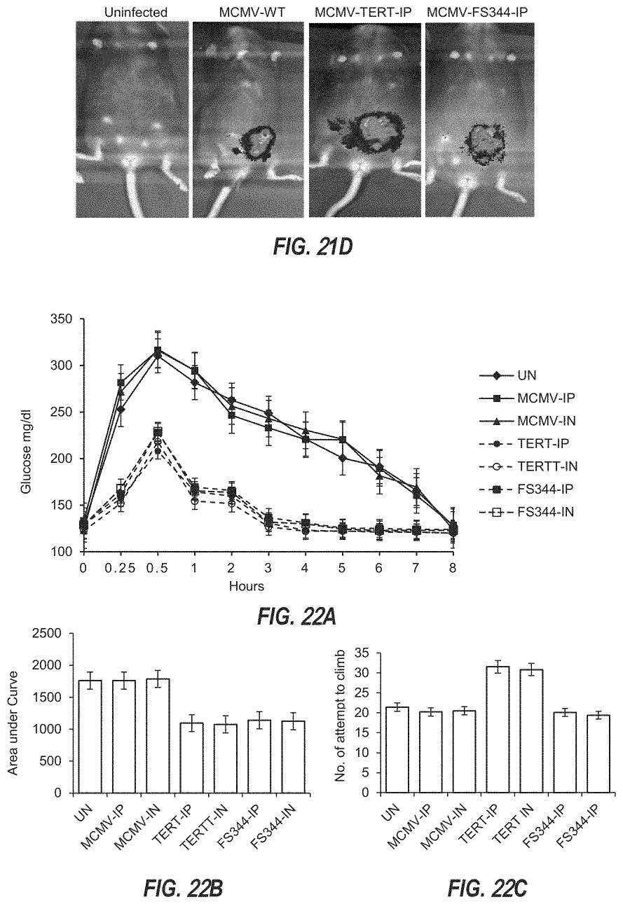

A BAC engineering method was used to generate a recombinant MCMV containing mouse telomerase reverse transcriptase (mTERT) gene as described previously (see Qiyi Tang, B. S., Hua Zhu. Protocol of a Seamless Recombination with Specific Selection Cassette in PCR-Based Site-Directed Mutagenesis. Applied Biological Engineering—Principles and Practice, 461-478 (2012)). Similarly, a recombinant MCMV containing the mouse follistatin (mFS344) gene was generated (FIG. 21A). The plasmids containing the mouse TERT gene (MR226892) and mouse follistatin gene (MR225488) were obtained from Origene. The mTERT and mFS344 were fused with a FLAG-tag at the C-terminus. The strategy to generate the recombinant MCMV is shown in FIG. 21A. A CMV promoter was used to express the mTERT and mFS344 genes due to its strong transcriptional activity. The mTERT and mFS344 expression cassette were inserted at M107 locus of the MCMV_{Luc} genome without any deletion using a single-step integration event. The integration of mTERT and mFS344 into the MCMV genome was confirmed by PCR, Western blot, and immunofluorescence assays. The recombinant MCMV_{Luc-TERT} and MCMV_{Luc-FS344} were characterized by Western blot using FLAG-tag monoclonal antibody to check the expression of mTERT and mFS344 in the infected cells.

The recombinant MCMV_{Luc} vector was characterized by growth curve, and Western blot. A growth curve was performed in mouse fibroblast 3T3 cells. The cells, in triplicate, in a six-well plate, were infected with 0.1 MOI of MCMV_{Luc-TERT} and MCMV_{Luc-Fs344}. The growth curves were determined by measuring the relative luciferase unit (RLU) every alternate day using luciferin substrate by In vivo Imaging System (IVISTM 50, Xenogen). Briefly, 3T3 cells were infected with the MCMV_{Luc-TERT} and MCMV_{Luc-FS344} cells at an MOI of 0.1. Two days later, the infected cells were harvested for Western blot analysis. Western blot results confirmed the expression of mTERT and mFS344 in the infected cells (FIG. 21B).

For Western blot analysis, 3T3 cells were infected with the MCMV_{Luc-TERT} and MCMV_{Luc-FS344} cells at an MOI of 0.2. Two days later, the infected cells were harvested and cell lysates were separated on 10% sodium dodecyl sulfate-polyacrylamide gel electrophoresis (SDS-PAGE) and proteins were transferred onto a PVDF membrane (Bio-Rad). The membrane was blocked with 3% BSA in 1X PBS for 2 hours at room temperature (RT) followed by 1-hour incubation with a mouse FLAG-tag antibody (1:2000 dilution in 3% BSA in 1X PBS). The membrane was washed three times with the 0.1% Tween-20 for 10 minutes each. Thereafter, the membrane was incubated with a secondary anti-mouse HRP conjugated antibody at a dilution of 1:10,000 in 1X BSA for 1 hour at RT. The blot was developed by chemiluminescence substrate (Bio-Rad).

Growth of MCMV_{Luc-TERT} and MCMV_{Luc-FS344} were determined using a luciferase assay. The growth curve analysis confirmed that MCMV_{Luc-TERT} and MCMV_{Luc-FS344} are similar to MCMV_{-WT} and do not show any growth defect (FIG. 21C). The in vivo replication of recombinant viruses in 19-month-old C57BL/6J mice also confirms that these viruses replicate as well as WT virus (FIG. 21D).

### Example 3: In Vivo Efficacy of MCMV_{Luc-TERT} and MCMV_{Luc-FS344} Recombinant Viruses in Old Mice

The therapeutic efficacy and safety of the disclosed and tested recombinant viruses was determined using 18-month old C57BL/6J mice. Two routes of viral administration were chosen in mice, namely intranasal (IN) and intraperitoneal (IP). The mice were separated into the following 7 groups with 9, 18-month old C57BL/6J mice per group: (1) MCMV_{Luc-WT-IN}, (2) MCMV_{Luc-WT-IP}, (3) Uninfected, (4) MCMV_{Luc-TERT-IP}, (5) MCMV_{Luc-TERT-IN}, (6) MCMV_{Luc-FS344-IP}, and (7) MCMV_{Luc-FS344-IN}. The mTERT and mFS344 expressing recombinant viruses were injected once a month, at a dose of 1×105 PFU/mouse.

### Example 4: Glucose Intolerance Test

Three mice from each group were selected at 22 months of age: (1) MCMV_{Luc-WT-IN}, (2) MCMV_{Luc-WT-IP}, (3) Uninfected, (4) MCMV_{Luc-TERT-IP}, (5) MCMV_{Luc-TERT-IN}, (6) MCMV_{Luc-FS344-IP}, and (7) MCMV_{Luc-FS344-IN}. The mice were starved for 15 hours, followed by intraperitoneal injection with a 50 mg glucose solution. Blood samples were collected at 0 min, 15 min, 30 min, 60 min, 120 min, 180 min, 240 min, 300 min, 360 min, 420 min, and 480 min via a small incision in the tail vein of the mice. The blood glucose level was immediately determined using OneTouch Ultra glucose strips.

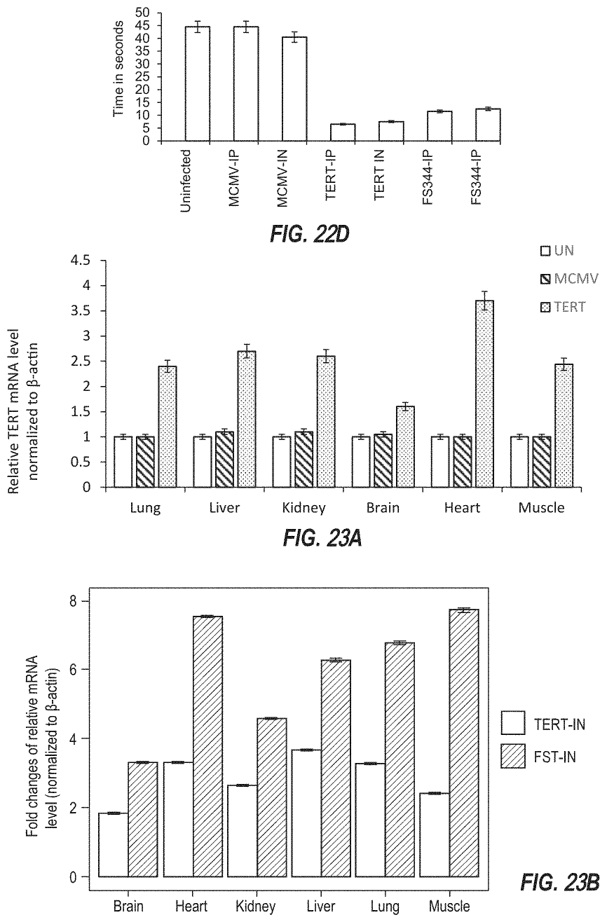

A peak of glucose level was observed at 30 minutes of glucose administration (FIG. 22A). After 30 minutes, the uninfected or WT treated mice were showing high blood glucose with an average value of 310 mg/dl and 316 mg/dl respectively, whereas the mTERT and mFS344 treated mice were showing 154 mg/dl and 165 mg/dl glucose. Moreover, blood glucose level came back to the normal basal level in 180 minutes in the mFS344 and mTERT treated mice. The WT or uninfected mice took about 480 minutes to bring blood glucose levels to the normal basal level (FIG. 22A). The area under curve (AUC) of the glucose curve is shown in FIG. 22B and confirms glucose intolerance of mice treated with mTERT and mFS34 recombinant viruses. Delivery, metabolism and expression of mTERT and mFS344 enables improved control over glucose metabolism and blood glucose levels in treated mice. Effectively controlling and/or treating blood glucose levels is an effective method of treating and/or preventing Type II Diabetes, which is known to be associated with elevated levels of blood glucose and an inability to metabolize glucose.

### Example 5: MCMV_{Luc}-TERT and MCMV_{Luc}-FS344 Infected Old Mice have Less Glycosylated Hemoglobin

The serum level of glycosylated hemoglobin is a direct measure of a defective metabolism and indicates diabetic mice. Serum from mice in each group was analyzed by the ELISA kit (M0476F041, Mybiosource) per the manufacturer's method to calculate the level of glycosylated hemoglobin.

The level of glycosylated hemoglobin is a direct measure of blood glucose level and its increase is indicative of diabetes. Here, we measured the level of glycosylated hemoglobin in 23-month-old mice after 5 consecutive administrations of respective therapy treatments. Mice infected with recombinant mTERT and mFS344 virus were found to contain less glycosylated hemoglobin in their blood as compared to the control or untreated mice. A high level of glycosylated hemoglobin is associated with diabetic conditions. This finding correlates well with the glucose tolerance test results, further supporting why MCMV_{Luc-TERT} and MCMV_{Luc-FS344} reduce blood glucose over 3 times faster than the control groups.

### Example 6: MCMV_{Luc-TERT} and MCMV_{Luc-FS344} Treated Old Mice have Increased Activity and Coordination

Body activity and coordination decrease progressively with age. A measure of activity was considered an indication of the overall health of a patient. Therefore, a beaker test was performed to determine the activity of the treated elderly mice in each group. This test was performed when mice were 23-months old. Three mice from each group were put in a one liter glass beaker and general activity and attempts to climb out of the beaker were measured. The protocols were properly followed as described previously (see, for example, Magno, et al. Optogenetic Stimulation of the M2 Cortex Reverts Motor Dysfunction in a Mouse Model of Parkinson's Disease. J Neurosci 39, 3234-3248, doi:10.1523/Jneurosci.2277-18.2019 (2019) and Magno, et al. Cylinder Test to Assess Sensory-motor Function in a Mouse Model of Parkinson's Disease. Bio-Protocol 9, doi:ARTN e3337 10.21769/BioProtoc.3337 (2019)).

MCMV_{Luc-TERT} treated animals were more active than uninfected or MCMV_{Luc-WT} treated mice (FIG. 22C). The MCMV_{Luc-FS344} treated mice were bulkier and were not able to climb but showed increased activity by walking faster on the floor of the beaker than the control or untreated mice. Surprisingly, mFS344 treated mice were more active in penetrating the bedding of cages than other groups, suggesting more power and energy.

Aging mice generally have reduced coordination and motor skills. The coordination of treated and untreated mice was analyzed using a beam walking assay as performed previously (see Luong, et al. Assessment of Motor Balance and Coordination in Mice using the Balance Beam. Jove-J Vis Exp, doi:10.3791/2376 (2011)). In short, three, 23-month old mice from each group were trained to traverse a 4 ft long, 10 mm wide beam for two consecutive days, then tested on the third day. Results were measured as the amount of time taken by each mouse to cross the beam. The mice treated with MCMV_{Luc-TERT} and MCMV_{Luc-FS344} exhibited better coordination as compared to the uninfected or MCMV_{Luc-WT} treated mice (FIG. 22D). These results confirmed that MCMV_{Luc-TERT} and MCMV_{Luc-FS344} treated mice have improved cognitive ability paired with enhanced activity and coordination, suggesting improved health in aging mice.

### Example 7: Tissues RNA Isolation, cDNA Preparation and Real-Time PCR

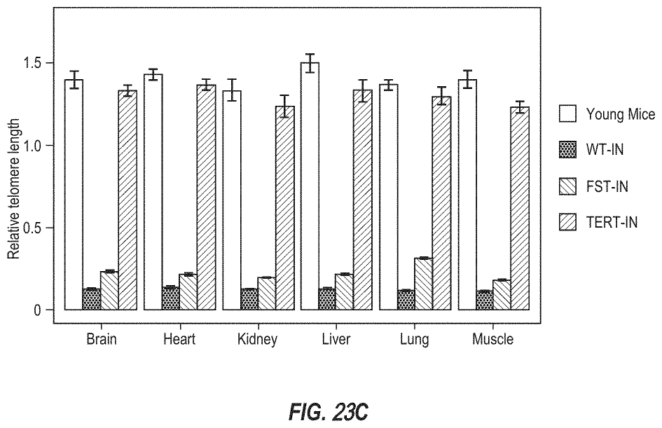

To determine the level of mTERT and mFS344 in various tissues of mice infected with MCMV_{Luc-TERT}, real-time PCR was performed on cDNA prepared from RNA of infected tissues. Eleven-month-old mice (3 in each group) were infected with wild type MCMV, MCMV_{Luc-TERT}, and MCMV_{Luc-Fs344}. The heart, brain, lung, liver, and kidney were isolated 6-days post infection. Uninfected mice were used as a control. The tissues were homogenized, and RNA was isolated using RNeasy® Mini Kit (Qiagen). Purified RNA concentration was determined by measuring optical density. Approximately, 1.0 μg of RNA was used to prepare cDNA using a Titanium RT-PCR kit (TaKaRa). Real-time PCR was performed on the cDNA using mTERT, and mFS344 primers as described previously. β-actin was used as an internal control for normalization. The expression of mTERT and mFS344 was determined in respective tissues after normalization with β-actin. Results for TERT mRNA levels are shown in FIG. 23A. The fold increase in mRNA levels for TERT and FST are shown in FIG. 23B. As shown, TERT mRNA was increased by ˜1.9, 3.3, 2.7, 3.7, 3.3, and 2.4 folds in brain, heart, kidney, liver, lung, and muscle respectively as compared to the control, while FST mRNA was increase by ˜3.3, 7.6, 4.6, 6.3, 6.8, and 7.8 folds in brain, heart, kidney, liver, lung, and muscle respectively as compared to the control (FIG. 23B).

### Example 8: Determination of Relative Telomere Length in Organs of Treated and Untreated Mice

FIG. 23C illustrates the determination of relative telomere length in different organs of treated and untreated groups at 24-months-old mice. An 8-month-old mouse was also measured. The qPCR was performed on the genomic DNA using specific telomeric primers. A pair of 36B4 gene primers was used as a single copy gene for normalization. As shown, the follistatin treated mice showed increased telomere length relative to untreated mice. The TERT treated mice showed even greater increases in telomere length relative to untreated mice. Surprisingly, the TERT treated mice showed telomere lengths approaching (or in some cases not statistically different from) the telomere lengths of the young (8-month-old) mice.

### Example 9: Serum Level of mTERT and mFS344 Protein in Old and Young Mice Infected with MCMV_{Luc-TERT} and MCMV_{Luc-FS344}

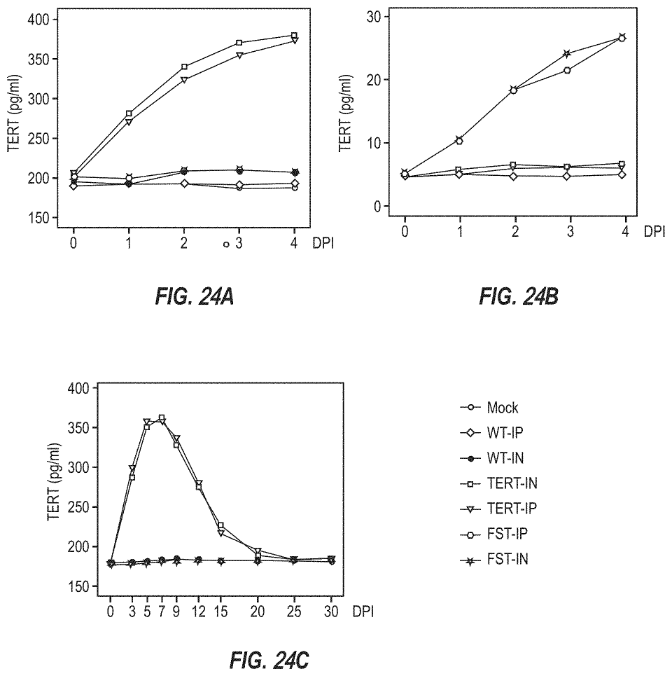

After 6 consecutive months of administration of recombinant MCMV_{TERT} and MCMV_{FS344} virus in 18-month-old mice, blood was analyzed for 3-4 days to determine the serum level of mTERT and mFS344. The collected blood was centrifuged, and serum was separated. A mouse telomerase reverse transcriptase ELISA kit (MBS1601022, Mybiosource) and a mouse follistatin ELISA kit (MBS1996306, MyBioSource) were used to measure the serum level of mTERT and mFS344, respectively. The protocol and instructions were followed as per the manufacturer's method. ELISA results demonstrate that there was an increase in the level of mTERT (380 pg/ml) and follistatin (26.6 ng/ml) in the serum after the administration of recombinant viruses compared to the control or MCMV infected (“Mock” treated) mice (FIGS. 24A & 24B). It confirms that MCMV_{Luc-TERT} and MCMV_{Luc-FS344} infect various cells types throughout the body and express the target proteins.

The experiment was repeated with 8-month old C57BL/6J mice to determine the kinetics of mTERT and mFS344 expression over one month (FIG. 24C). Three mice in each group were infected with MCMV_{Luc-TERT} and blood samples were collected at indicated time points. The serum mTERT level was highest (˜400 ng/mL) at 5-7 days post-infection. After 7 days of infection, the expression of mTERT decreases gradually, reaching basal level by day 25 (FIG. 24C).

### Example 10: MCMV_{Luc-TERT} and MCMV_{Luc-FS344} Infected Old Mice Look Younger with Less Hair Loss

Hair loss was observed in each group of mice. MCMV_{Luc-TERT} and MCMV_{Luc-FS344} treated mice were observed for body hair loss over time. The mice treated with a wild-type MCMV vector or left untreated were used as controls. High-resolution photographs were captured and analyzed to determine the degree of hair loss in each mouse. The pictures in FIGS. 5A-5D are of 24-month old mice that are a representative specimen of their respective groups.

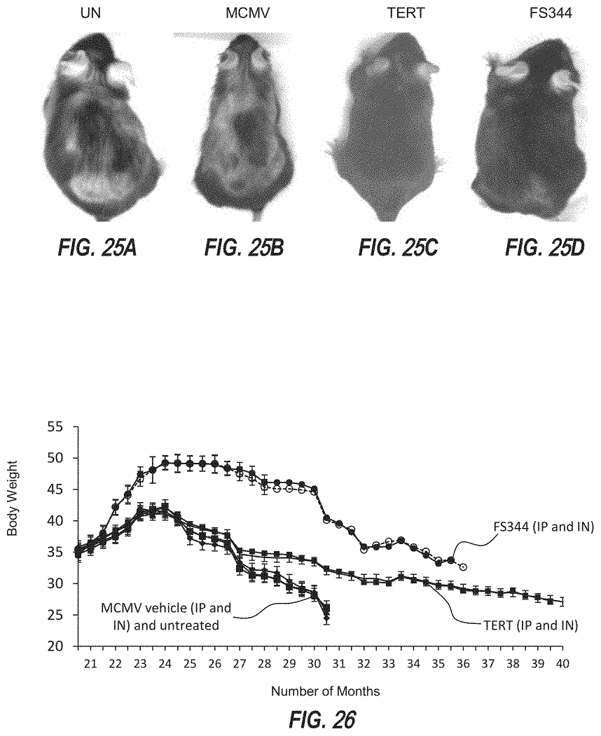

MCMV_{Luc-TERT} treated animals were resistant to hair loss and retained 99 percent of their body hair after 26 months. At this time point, the mice in each group were administered with respective viral vectors 8 times for 8 months of anti-aging therapy. The untreated and MCMV_{Luc-WT} treated mice were losing their hair rapidly and retained only 40 percent of the hair (FIG. 25B). MCMV_{Luc-TERT} (FIG. 25C) and MCMV_{Luc-FS344} (FIG. 25D) treated animals were looking younger as compared to the uninfected (FIG. 25A) and MCMV_{Luc-WT} (FIG. 25B) treated animals and did not lose their hair after a period of 26 months. These results strongly suggest that mice infected with MCMV_{Luc-TERT} and MCMV_{Luc-FS344} were more resistant to hair loss and looked younger, while the mice in untreated or MCMV_{Luc-WT} treated cages looked older and lost their hair continuously as they aged. The loss of hair may be correlated with the TERT expression in the skin cells, as some studies have hinted at an increase in TERT expression within skin as potentially facilitating hair growth by enhancing the follicle stem cells proliferation. Unexpectedly, however, the mice infected with recombinant MCMV_{Luc-FS344} virus also demonstrated a similar retention of hair as the MCMV_{Luc-TERT} infected mice.

### Example 11: MCMV_{Luc-TERT} and MCMV_{Luc-FS344} Infected Old Mice have Increased Bodyweight

The bodyweight of all mice in each group was measured and recorded twice a month. An increase in body mass was considered to be a measure of accumulation of more myocytes in the body. The mice in each set were analyzed postmortem for their increases in muscle mass and muscle gain.

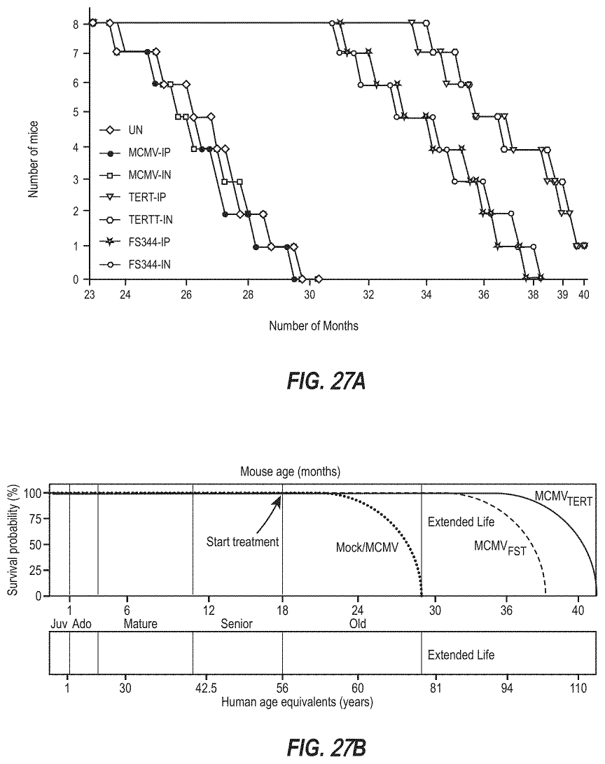

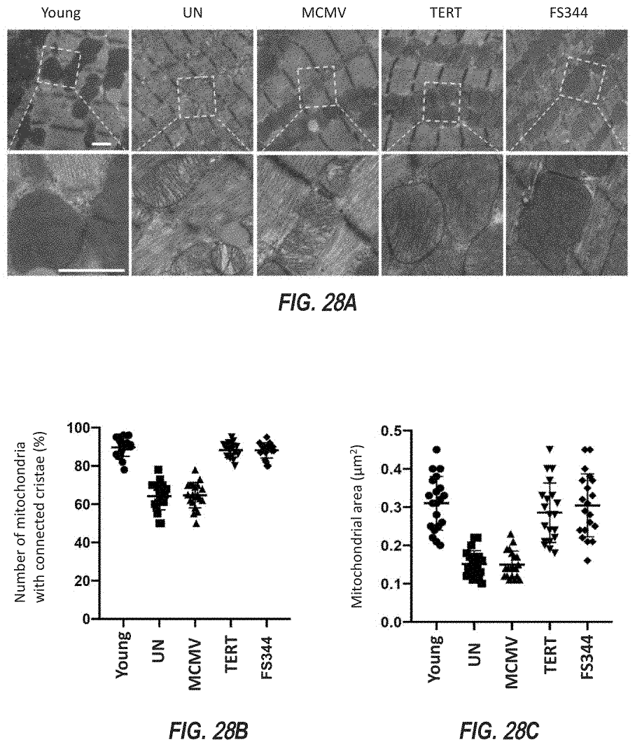

Recombinant viruses were injected once a month into each group of 18-month old C57BL/6J mice to determine therapeutic efficacy. The bodyweight of all mice in each group was measured twice a month to see any effect of recombinant viruses on normal health. An increase in body mass was interpreted as an accumulation of myocytes and increase in musculature. MCMV_{Luc-FS344} treated mice weighed ˜40% more than MCMV_{Luc-WT} treated mice. The average body weight of MCMV_{Luc-FS344} treated mice was ˜49 gm after 150 days of treatment (FIG. 26). The uninfected and MCMV_{Luc-WT} treated mice have an average weight of ˜35 gm and ˜37 gm after 150 days of treatment. The MCMV_{Luc-TERT} infected mice had an average body weight of ˜39 gm and showed an overall resistance in body weight loss (FIG. 26). Bodyweight analysis suggested an increase in body weight for MCMV_{Luc-FS344} treated mice as compared to MCMV_{Luc-WT} treated or uninfected groups. The MCMV_{Luc-TERT} infected animals substantially gained bodyweight and showed resistance in losing body weight with age.

Administration of MCMV_{Luc-TERT} and MCMV_{Luc-FS344} was stopped after mice reached 29 months of age. Interestingly, mice started losing weight (FIG. 26). This could be due to waning expression of mTERT and mFS344. After 32 months, therapy was reinitiated and an increase in body weight was measured, likely due to the expression of mFS344gene (FIG. 26). These results suggest a dose dependent increase in bodyweight of mice infected with MCMV_{Luc-Fs344}.

### Example 12: MCMV_{Luc-TERT} and MCMV_{Luc-FS344} Treated Old Mice have Increased Life Span

A Kaplan-Meier test was used to determine the longevity of MCMV_{Luc-TERT} and MCMV_{Luc-FS344} treated subjects over a given interval of time. The control animals died earlier than MCMV_{Luc-FS344} or MCMV_{Luc-TERT} animals. A significant life extension in MCMV_{Luc-TERT} and MCMV_{Luc-FS344} treated mice was observed as compared to the control. The average age of mice in MCMV_{Luc-WT-IP}, MCMV_{Luc-WT-IN}, and the uninfected group were 26.4, 26.6, and 26.6 months, respectively (FIGS. 27A and 27B). The average age of mice in MCMV_{Luc-FS344-IN} and MCMV_{Luc-FS344-IP} was 34.3 and 34.6 months, respectively (FIGS. 27A and 27B). MCMV_{Luc-TERT-IN} and MCMV_{Luc-TERT-IP} treated mice have an average age of 37.1 and 37.3 months, respectively (FIGS. 27A and 27B). A percentage increase in the longevity of mice in MCMV_{Luc-TERT} (41.1%) and MCMV_{Luc-FS344} (30.3%) infected groups was determined compared with the MCMV_{Luc-WT} infected or untreated mice. A significant increase in the median age of the mice in MCMV_{Luc-TERT} and MCMV_{Luc-FS344} treated mice was also observed as compared to the control groups (FIGS. 27A and 27B).

These results demonstrate that MCMV_{Luc-FS344} treated mice have a 30.3 percent increase in age longevity as compared to the control or untreated mice (FIGS. 27A and 27B). The mice infected with MCMV_{Luc-TERT} have a 40.1 percent increase in the age as compared to MCMV_{Luc-WT} infected or untreated groups (FIGS. 27A and 27B). These results strongly indicate overall longevity for MCMV_{Luc-TERT} and MCMV_{Luc-FS344} treated mice as compared to the uninfected or MCMV_{Luc-WT} treated mice. The significant increase in longevity alongside increased activity and coordination indicated improved health in old mice. This confirms that the CMV vector is able to infect a wide variety of cells in the body and thus is able to deliver the mTERT and mFS344 genes that lead to the significant increase in life span with increased coordination and body activity.

### Example 13: Old Mice Treated with MCMV_{Luc-TERT} and MCMV_{Luc-FS344} have Higher Mitochondria Number with Increased Cristae Surface Area in Heart and Skeletal Muscle Tissue

It has been demonstrated previously that accelerated aging produced by shortening of telomeres over time involves mitochondrial deterioration, suggesting a connection between telomerase and mitochondria in the aging process. Mitochondrial dysfunction is associated with aging, as cellular metabolism is impaired.

To observe changes occurring at the cellular level, electron microscopy was performed on tissue samples collected from animals in each set. One mouse from each group was sacrificed after 6 consecutive months of recombinant virus administration, and the heart and skeletal muscles were isolated. The tissue samples were fixed in EM buffer and kept at 4° C. until processed. Conventional EM was performed as known in the art.

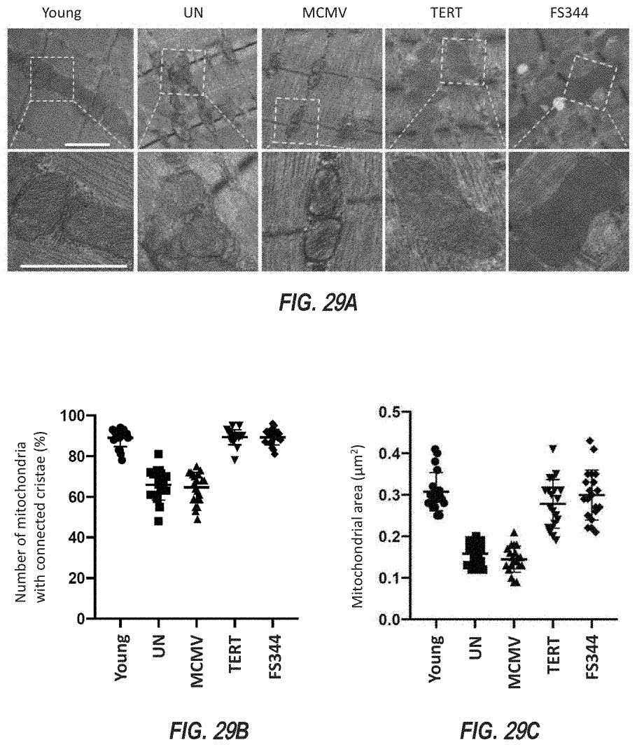

The electron microscopy analysis of heart and skeletal muscle tissues from MCMV_{Luc-TERT} and MCMV_{Luc-mFS344} treated mice have an increased number of mitochondria with connected cristae in heart and skeletal muscle tissue, comparable to 6 months old controls (FIGS. 28A, 28B, 29A, and 29B). The cristae are folds of the mitochondrial inner membrane that provide more surface area, allowing for an increase in the various processes carried out in/by the mitochondria. For example, the cristae allows for increased ATP production due to the increased surface area (i.e., there is more room for more ATP-producing reactions to occur). Connected and intact cristae is vital for these processes to occur. Impaired or disconnected cristae leads to impaired respiration processes and fragmentation of the mitochondrial network. Impaired cristae is also strongly implicated in the pathogenesis of human diseases.

The mice in the control group (MCMV and UN) have fewer mitochondria overall (FIGS. 28A, 28B, 29A, and 29B). The calculated surface area of heart tissue mitochondria in mice infected with MCMV_{Luc-TERT} and MCMV_{Luc-FS344} increased and was comparable to the young mice (FIGS. 28C and 29C).

### Example 14: Autophagy and Antiaging Markers

To address the mechanism behind the observed increased number mitochondria and their surface area, the level of PGC1α, a transcription coactivator required for the proper mitochondrial function and regulation of cellular signaling pathways controlling cell aging, was measured. Mitochondria and autophagy play a critical role in the biological aging process. The accumulation of dead or aging cells leads to cellular and systemic dysfunction, and thus causes aging. To determine the exact changes in the body of treated old mice, the protein level of various integral markers of autophagy and aging were measured. Western blot was used to determine the level of PGC1α (Novus Bio, NBP1-04676), Complex I, II, III, and V (Thermofisher, 45-8099), TFAM (Abcam, ab131607), LC3 (BML,M183-3), and p62 (Abcam, ab91526) markers in the tissue homogenates of treated and control mice. The specific monoclonal antibodies were used for specific markers.

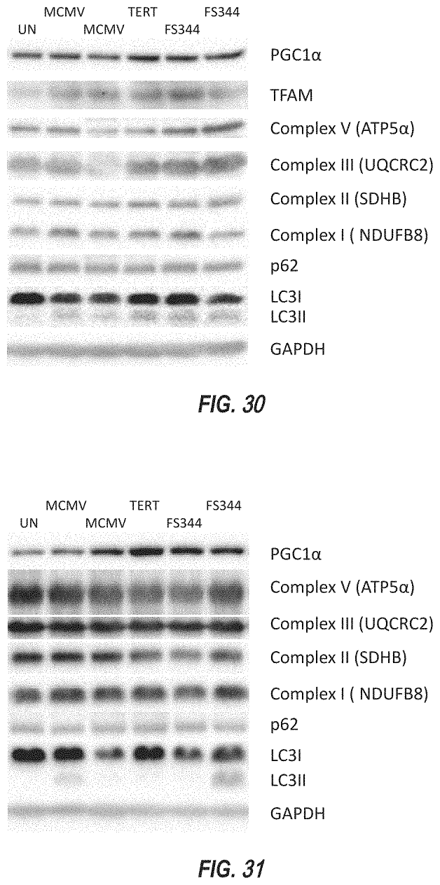

The MCMV_{Luc-TERT} and MCMV_{Luc-FS344} treated mice show increased autophagy markers by Western blot, suggesting the active removal of aging cells at a faster rate than the control group. The protein level of Complex I, II, III and V, PGC1α, TFAM, LC3I, LC3II, and p62 markers were specifically determined in the skeletal muscle tissue homogenates of treated and control mice (FIG. 30). The level of coactivator transcription factor PGC1α and TFAM (mitochondria transcription factor A) was found to be elevated in the skeletal muscles as compared to the control and untreated mice, suggesting increased mitochondrial biogenesis and other cellular pathways associated with muscle fiber development (FIGS. 30 and 31). An increased level of complex III (UQCRC2) in MCMV_{Luc-TERT} and MCMV_{Luc-FS344} treated mice was observed as compared to the control or untreated groups (FIGS. 30 and 31). Complex III is an essential component of mitochondria and is involved in ATP synthesis as well as oxygen homeostasis.

### Example 15: MCMV_{Luc-TERT} and MCMV_{Luc-FS344} Treated Old Mice Did not Show Incidence of Cancer

Because TERT activity increases in cancer cells, there is a concern that induced overexpression of mTERT may increase the risk of tumor or cancer development. However, no malignancies were detected upon autopsy in either treated group. Additionally, ante mortem daily observations did not reveal any other defects such as paralysis, body dysfunctions, or blindness. Notably, re-expressing telomerase in a model of increased aging could not detect cancer. These results demonstrate that overexpression of mTERT in old mice in the context of a recombinant CMV vector does not cause any cancer phenotype or malignancies, nor any other obvious systemic dysfunction.

In conclusion, mice infected with the recombinant TERT or FS344 virus show an increase in body weight, high glucose tolerance, high serum level of TERT and FS344, increased activity, and other anti-aging characteristics. The treated mice have younger mitochondria in heart and muscle tissues and an increased level of PGC1α which controls the expression of genes that cause aging. Moreover, the treated mice show an increased level of complex III, V, TFAM, and 1C3II leading to the anti-aging effect on old mice. These results demonstrate that CMV is an excellent vector for delivering the therapeutic TERT and/or FS344 proteins in patients and has immense potential for gene therapy.

1. A method for gene therapy using intranasal administration of genetically modified viral vectors, comprising:
creating a gene cassette comprising one or more donor genes;
inserting the gene cassette into a cloning vector and transfecting the cloning vector into a bacterial cell;
preparing a human-administrable viral vector to include the one or more genes, wherein the human-administrable viral vector is a human cytomegalovirus or a varicella zoster virus;
preparing a solution containing a therapeutic amount of viral agents comprising the plurality of target genes; and
administering the solution to the subject.

2. The method of claim 1, wherein the solution is administered intranasally.

3. The method of claim 1, wherein the solution is administered via injection.

4. The method of claim 1, wherein the virus is a human cytomegalovirus.

5. The method of claim 1, wherein the one or more genes comprise hTERT.

6. The method of claim 1, wherein the one or more genes comprise mTERT.

7. The method of claim 1, wherein the one or more genes comprise hFS344.

8. The method of claim 1, wherein the one or more genes comprise mFS344.

9. The method of claim 1, wherein the one or more genes comprise both TERT and FS344.

10. The method of claim 9, wherein the one or more genes comprise both hTERT and hFS344.

11. The method of claim 1, wherein the subject is a human.

12. The method of claim 1, wherein the subject is a laboratory animal.

13. The method of claim 12, wherein the subject is a mouse.

14. The method of claim 1, wherein the cloning vector is a plasmid.

15. A method for gene therapy using intranasal administration of genetically modified viral vectors, comprising:
creating a gene cassette comprising one or more donor genes, the one or more donor genes comprising hTERT, hFS344, or both;
inserting the gene cassette into a cloning vector and transfecting the cloning vector into a bacterial cell;
preparing a human-administrable viral vector to include the one or more genes, wherein the human-administrable viral vector is a human cytomegalovirus;
preparing a solution containing a therapeutic amount of viral agents comprising the plurality of target genes; and
administering the solution to the subject.

16. The method of claim 15, wherein the subject is a human.

17. The method of claim 15, wherein the solution is administered intranasally.

18. The method of claim 15, wherein the solution is administered via injection.

19. The method of claim 15, wherein the one or more genes comprise both hTERT and hFS344.

20. A method for gene therapy using intranasal administration of genetically modified viral vectors, comprising:
creating a gene cassette comprising both hTERT and hFS344;
inserting the gene cassette into a cloning vector and transfecting the cloning vector into a bacterial cell;
preparing a human-administrable viral vector to include the one or more genes, wherein the human-administrable viral vector is a human cytomegalovirus;
preparing a solution containing a therapeutic amount of viral agents comprising the plurality of target genes; and
administering the solution to a human subject.
